SEQUENCE LISTING (9 sequences)

SEQ ID NO: 1 - protein, 41 aa, influenza virus
PPNSKEQQNLYQNENAYVSVVTSNYNRRFTPEIAERPKVRD

SEQ ID NO: 2 - protein, 41 aa, synthetic construct
PPNSKEQLGLYGNANAYVSVVTSNYNRRFTPEIAERPKVRD

SEQ ID NO: 3 - protein, 41 aa, synthetic construct
PPNSKEQFVLYSNANAYVSVVTSNYNRRFTPEIAERPKVRD

SEQ ID NO: 4 - protein, 41 aa, synthetic construct
PPNSKEQVRLYGNINAYVSVVTSNYNRRFTPEIAERPKVRD

SEQ ID NO: 5 - protein, 41 aa, synthetic construct
PPNSKEQNELYLNANAYVSVVTSNYNRRFTPEIAERPKVRD

SEQ ID NO: 6 - protein, 41 aa, synthetic construct
PPNSKEQMVLYLNDNAYVSVVTSNYNRRFTPEIAERPKVRD

SEQ ID NO: 7 - protein, 41 aa, synthetic construct
PPNSKEQDLLYPNGNAYVSVVTSNYNRRFTPEIAERPKVRD

SEQ ID NO: 8 - protein, 41 aa, synthetic construct
PPNSKEQTTLYHNVNAYVSVVTSNYNRRFTPEIAERPKVRD

SEQ ID NO: 9 - protein, 41 aa, synthetic construct
PPNSKEQNRLYPNTNAYVSVVTSNYNRRFTPEIAERPKVRD

(19) United States
(12) Patent Application Publication
(10) Pub. No.: US 2025/0144198 A1
(43) Pub. Date: May 8, 2025

(54) NEXT GENERATION VACCINES COMPRISING ANTIGENIC LIBRARIES AND METHODS OF MAKING AND USING SAME

(71) Applicant: Duke University, Durham, NC (US)

(72) Inventors: Nicholas HEATON, Durham, NC (US); Zhaochen LUO, Durham, NC (US)

(21) Appl. No.: 18/684,346

(22) PCT Filed: Aug. 16, 2022
(86) PCT No.: PCT/US2022/075030

§ 371 Date: Feb. 16, 2024

Related U.S. Application Data
(60) Provisional application No. 63/233,361, filed on Aug. 16, 2021.
(60) Provisional application No. 63/392,047, filed on Jul. 25, 2022.

(51) Int. Cl. A61K 39/145; A61P 37/04; C07K 14/005; C12N 15/10; G01N 33/68

(52) U.S. Cl. CPC A61K 39/145; A61P 37/04; C07K 14/005; C12N 15/1058; G01N 33/6845; C12N 2760/16022; C12N 2760/16034; C12N 2760/16071

(57) ABSTRACT

The present invention provides protein libraries comprising variants of an antigenic viral protein that each have a different set of mutations at one or more hypervariable sites. Nucleic acid and vector libraries encoding the protein libraries, vaccines comprising the libraries, and methods of inducing an immune response against the antigenic viral protein are also provided.

### CROSS-REFERENCE TO RELATED APPLICATIONS

This application claims priority to U.S. Provisional Application No. 63/233,361 filed on Aug. 16, 2021, and to U.S. Provisional Application No. 63/392,047 filed on Jul. 25, 2022, the contents of both of which are incorporated herein by reference in their entirety.

### STATEMENT REGARDING FEDERALLY SPONSORED RESEARCH

This invention was made with government support under grant number 75N93019C0005 awarded by the National Institutes of Health Division of Microbiology and Infectious Disease (NIH/NIAID/DMID). The government has certain rights in this invention.

### SEQUENCE LISTING

A Sequence Listing accompanies this application and is submitted as an XML file of the sequence listing named “155554.00663.xml” which is 8,935 bytes in size and was created on Aug. 10, 2022. The sequence listing is electronically submitted via Patent Center with the application and is incorporated herein by reference in its entirety.

### BACKGROUND

Influenza imposes a large burden on human health. The World Health Organization estimates that influenza infects approximately 1 billion people worldwide annually, of which 3-5 million cases are severe and 300,000-500,000 people die.

The influenza glycoprotein hemagglutinin (HA) is the most important antigen for stimulating a protective immune response following infection or vaccination. Many of the key antigenic sites on HA (i.e., sites that neutralizing antibodies are generated against) are hypervariable. As a result, flu vaccines must be updated annually to provide protection against new influenza variants in which these hypervariable sites have mutated.

It takes time to manufacture a vaccine. Thus, scientists must predict which specific viral strains will cause significant disease prior to the start of the flu season and develop vaccines based on these predictions. Because influenza viruses mutate quickly, new strains that evade the immune response produced by the vaccines developed that year often arise during a flu season. Thus, there is a need in the art for universal influenza vaccines that can protect against highly drifted influenza viruses.

### SUMMARY

In a first aspect, the present invention provides protein libraries comprising variants of an antigenic viral protein. Each variant of the antigenic viral protein comprises a different set of mutations at one or more hypervariable sites.

In a second aspect, the present invention provides nucleic acid libraries encoding the protein libraries disclosed herein. Each nucleic acid in the nucleic acid library encodes one of the variants of the antigenic viral protein of the protein library.

In a third aspect, the present invention provides vector libraries comprising a nucleic acid library disclosed herein. Each vector in the vector library comprises one of the nucleic acids of the nucleic acid library.

In a fourth aspect, the present invention provides virus libraries comprising a vector library or nucleic acid library disclosed herein. Each virus in the virus library comprises one of the vectors of the vector library or one of the nucleic acids of the nucleic acid library.

In a fifth aspect, the present invention provides vaccines comprising a protein library, nucleic acid library, vector library, or virus library described herein. The vaccines may also include a pharmaceutically acceptable carrier, diluent, or adjuvant.

In a sixth aspect, the present invention provides methods of inducing an immune response against the antigenic viral protein in a subject. The methods comprise: administering to the subject a protein library, nucleic acid library, vector library, virus library or vaccine disclosed herein.

In a seventh aspect, the present invention provides methods of generating a vector library described herein. The methods comprise: (a) performing saturation mutagenesis on nucleic acids encoding the antigenic viral protein at one or more hypervariable sites of the antigenic viral protein to obtain mutated nucleic acids; (b) cloning the mutated nucleic acids into viral vectors; (c) packaging the viral vectors into viruses; (d) transducing the viral vectors into cells by infecting the cells with the viruses; (e) identifying infected cells that express the antigenic viral protein; and (f) cloning the mutated nucleic acids encoding variants of the antigenic viral protein found in the infected cells into an expression vector to generate the vector library.

In an eighth aspect, the present invention provides methods of performing saturation mutagenesis on a plurality of target sites within a DNA sequence of interest. The methods comprise: (a) designing a plurality of overlapping primers that are complementary to sequences flanking the target sites and comprise mutations at the target sites; (b) performing a recombination reaction in which the primers are annealed to each other to form a set of recombinant primers that each comprise a different set of mutations at the target sites; and (c) extending the recombinant primers using a DNA polymerase to form full-length sequences that span the target sites; (d) amplifying the full-length sequences to form an amplification reaction product; (e) purifying the amplification reaction product; and (f) cloning the amplification reaction product into an appropriate vector.

### BRIEF DESCRIPTION OF THE DRAWINGS

FIG. 1 depicts the design, generation, and characterization of an antigenically complex hemagglutinin (HA) vaccine. (A) Model of H1 HA trimer. Residues that were mutated are indicated in orange. The model is based on the published crystal structure of the HA of A/Puerto Rico/8/1934, PDB number 5VLI (Nature 550:74-79, 2017). (B) Schematic depiction of the methods used to generate the mutant library. (C) Percentage of 6F12 positive cells in the Sb mutant HA library determined by flow cytometry after MACS. (D) Sequencing results for eight clones from the sorted library. (E) Glycosylation pattern of the Sb mutant HA library determined by SDS-PAGE. (F) Antigenicity of the Sb mutant HA library determined by ELISA with monoclonal antibodies targeting different antigenic sites (n=3). The statistical differences for panel F were determined using unpaired Student's t test. Horizontal bars indicate mean values. *, p<0.05; **, p<0.001; ns, not significant.

FIG. 2 demonstrates that vaccination with antigenically complex hemagglutinin proteins induces an increased number of functional head- and stalk-directed antibodies. (A) Schematic depiction of the vaccination regimen. Mice received two doses of vaccine, three weeks apart. Sera was collected three weeks after boost for analysis. Antibody response to PR8 HA is depicted as curve (B) or area under the curve (AUC) (C). Antibody response to PR8 virus is depicted as curve (D) or AUC (E). (F) Diagram of full-length HA, HA head, and HA stalk. AUC of the antibody response to PR8 HA head (G) and stalk (H). (I) Ratio of AUC of PR8 HA head to full-length PR8 HA. (J) Ratio of AUC of PR8 HA stalk to full length PR8 HA. N=10. The statistical differences for panels C, E, G, and H were determined using one-way ANOVA followed by Tukey's post hoc analysis. The statistical differences for panels I and J were determined using a Student's t test. For all panels, horizontal bars indicate mean values, error bars represent the standard error of the mean (SEM).

FIG. 3 demonstrates that vaccination with antigenically complex hemagglutinin proteins provides improved, and at least partially antibody mediated, protection from homologous challenge. Mice received two doses of vaccine, three weeks apart. Sera was collected three weeks after boost for hemagglutinin inhibition (HAI) analysis (A-B) and neutralization activity analysis (C) (n=10). Then mice were intranasally infected with PR8 virus 21 days after boost (D). Body weight (E) and survival (F) were monitored and recorded for 14 days post infection (n=10). Alternatively, mice were primed with vaccine and were intranasally infected with PR8 virus 21 days after prime (G). Body weight (H) and survival (I) were monitored and recorded until 14 days post infection (n=9). Lungs were collected at 4 days post infection for lung H&E staining (J) (representative lung lobes from each group are shown, n=3) or at 3 days post infection for virus detection (K) (n=5). Bodyweight change and survival after viral challenge were analyzed by two-way ANOVA followed by Sidak's multiple-comparison test or a log rank (Mantel-Cox) test, respectively. Statistical differences for panels B, C, and K were determined using one-way ANOVA followed by Tukey's post hoc analysis. For all panels, horizontal bars indicate mean values. Error bars represent the standard error of the mean (SEM). *, p<0.05; **, p<0.001.

FIG. 4 shows responses to heterologous strains. (A) H1 HA Luminex assay. Mice received two doses of vaccine, three weeks apart. Sera was collected three weeks after prime and three weeks after boost for analysis (n=5). (B-F) ELISA AUC of boost sera against H1N1 viruses (n=10). Antibody function analysis including HAI (G and H) (n=10), neutralization activity (I) (n=6 or 7), and ADCC reporter assay (J) (n=10) against Cal09 virus after boost. Statistical differences for panels B, C, D, E, and F were determined using one-way ANOVA followed by Tukey's post hoc analysis. Statistical differences in panel J were analyzed by two-way ANOVA followed by Sidak's multiple-comparison test. For all panels, horizontal bars indicate mean values, and error bars represent the standard error of the mean (SEM). *, p<0.05.

FIG. 5 demonstrates that antigenically complex hemagglutinin vaccination provides better protection against Cal09 challenge. Mice received two doses of vaccine, three weeks apart. Then mice were intranasally infected with Cal09 virus 21 days after boost (A). Body weight (B) and survival (C) were monitored and recorded for 14 days post infection (n=10). Alternatively, mice were primed with vaccines and were intranasally infected with Cal09 virus 21 days after prime (D). Body weight (E) and survival (F) were monitored and recorded until 14 days post infection (n=5). Mice were primed and then infected with Cal09 virus, lungs were collected at 6 days post infection for lung H&E staining (G) (representative lung lobes from each group are shown, n=3) or virus detection (H) (n=5). Bodyweight change and survival after viral challenge were analyzed by two-way ANOVA followed by Sidak's multiple-comparison test or a log rank (Mantel-Cox) test, respectively. Statistical differences for panel H were determined using one-way ANOVA followed by Tukey's post hoc analysis. For all panels, horizontal bars indicate mean values, and error bars represent the standard error of the mean (SEM). *, p<0.05; **, p<0.001.

FIG. 6 shows the antigenicity of the head-only and stalk-only HA constructs. Antigenicity of HA constructs was determined by ELISA with monoclonal or polyclonal antibodies targeting different epitopes (n=4). Statistical differences were determined using unpaired Student's t test. Horizontal bars indicate mean values. *, p<0.05; **, p<0.001; ns, not significant.

FIG. 7 demonstrates that priming with antigenically complex hemagglutinins induces an increased number of functional head- and stalk-directed antibodies. Mice received one dose of vaccine and sera was collected 21 days post prime for analysis. Antibody response to PR8 HA is depicted as a curve (A) or AUC (B). Antibody response to PR8 virus is depicted as a curve (C) or AUC (D). AUC of antibody response to PR8 HA head (E) and stalk (F). (G) Ratio of AUC of PR8 HA head to full-length PR8 HA. (H) Ratio of AUC of PR8 HA stalk to full-length PR8 HA. N=10. Statistical differences for panels B, D, E, and F were determined using one-way ANOVA followed by Tukey's post hoc analysis. Statistical differences for panels G and H were determined using a Student's t test. For all panels, horizontal bars indicate mean values, and error bars represent the standard error of the mean (SEM).

FIG. 8 shows a functional analysis of antibodies against PR8 virus after prime. Mice received one dose of vaccine, and sera was collected three weeks later for HAI analysis (A) (n=10), neutralization activity analysis (B) (n=10), and ADCC reporter assay (C) (n=8). Statistical differences for panels A and B were determined using one-way ANOVA followed by Tukey's post hoc analysis. Statistical differences in panel C were analyzed by two-way ANOVA followed by Sidak's multiple-comparison test. For all panels, horizontal bars indicate mean values, and error bars represent the standard error of the mean (SEM). **, p<0.001.

FIG. 9 shows IgG isotyping of immune sera. Mice received two doses of vaccine, three weeks apart. Sera was collected three weeks after boost for analysis of ELISA AUC of IgG isotypes (A-C) and AUC ratio of IgG2b/IgG1 (D) and IgG2c/IgG1 (E). Alternatively, mice received one dose of vaccine and sera were collected three weeks after prime for analysis of ELISA AUC of IgG isotypes (F-H) and AUC ratio of IgG2b/IgG1 (I) and IgG2c/IgG1 (J). N=10. Statistical differences for panels A-C and F-H were determined using one-way ANOVA followed by Tukey's post hoc analysis. Statistical differences for panels D, E, I, and J were determined using unpaired Student's t test. Horizontal bars indicate mean values.

FIG. 10 shows homologous protection from different infectious challenge inoculums. Mice were primed with vaccine and were intranasally infected with different infectious challenge inoculums of PR8 virus 21 days after prime. N=5.

FIG. 11 shows the results of a passive transfer experiment with prime antisera. (A) Mice received sera from vaccine primed mice and were then challenged with PR8 virus 24 hours later. Body weight (B) and survival (C) were monitored and recorded until 14 days post infection. Cumulative data of two independent experiments (n=10) are shown. Bodyweight change and survival after viral challenge were analyzed by two-way ANOVA followed by Sidak's multiple-comparison test or a log rank (Mantel-Cox) test, respectively. Error bars represent the standard error of the mean (SEM). **, p<0.001.

FIG. 12 shows the results of an H3 HA Luminex assay. Mice received two doses of vaccine, three weeks apart. Sera was collected three weeks after prime and three weeks after boost for analysis (n=5).

FIG. 13 shows responses to heterologous strains after prime vaccination. (A-E) ELISA AUC of prime sera against H1N1 viruses. Antibody function analysis, including HAI (F), neutralization activity (G), and ADCC reporter assay (H) against Cal09 virus after prime. N=10. Statistical differences for panel A, B, C, D, E, and G were determined using one-way ANOVA followed by Tukey's post hoc analysis. Statistical differences in panel H were analyzed by two-way ANOVA followed by Sidak's multiple-comparison test. For all panels, horizontal bars indicate mean values, and error bars represent the standard error of the mean (SEM).

FIG. 14 is a schematic showing the hypervariable sites found in the HA proteins expressed by three different influenza subtypes, i.e., influenza A/H1, influenza A/H3, and influenza B.

FIG. 15 is a schematic depicting a novel method for performing saturation mutagenesis at several target sites within a sequence-of-interest. This method utilizes a pool of overlapping primers that collectively span the sequence-of-interest. The primers comprise mutations at the target sites but are complementary to the sequences surrounding the target sites. The primers are annealed to each other to form variants of the sequence-of-interest that comprise random combinations of different mutations at each of the target sites. Then, the single-stranded regions are filled in using DNA polymerase and the variant sequences are amplified via polymerase chain reaction (PCR).

FIG. 16 is a graphical abstract of the experiments and results described in the Examples. Because of immunodominance of hypervariable antigenic sites, most of the antibodies induced by wild-type HA target these sites and very few of the antibodies target conserved epitopes of HA. The antigenically complex Sb mutant HA library, in which the immunodominant site Sb was mutated by saturation mutagenesis, induces an increased number of antibodies that target conserved epitopes of HA.

### DETAILED DESCRIPTION

The present invention provides protein libraries comprising variants of an antigenic viral protein that each have a different set of mutations at one or more hypervariable sites. Nucleic acid and vector libraries encoding the protein libraries, vaccines comprising the libraries, and methods of inducing an immune response against the antigenic viral protein are also provided.

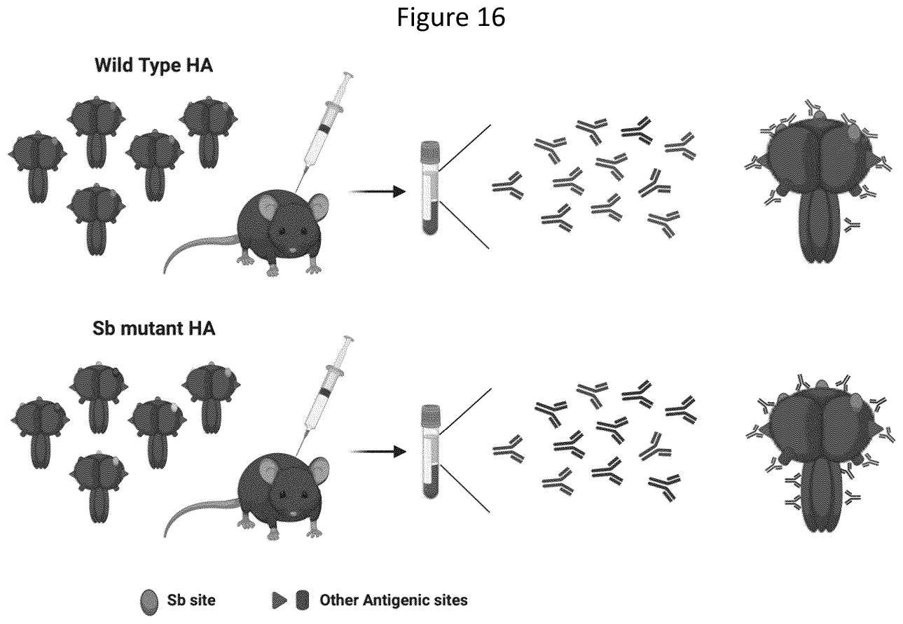

The present inventors have generated novel vaccines comprising a library of variants of an antigenic influenza protein. Specifically, the inventors have generated a vaccine comprising variants of a hemagglutinin (HA) protein comprising randomized amino acids at four positions in the antigenic site referred to as Sb. This library of variant proteins is referred to herein as “the Sb mutant HA protein mixture,” “the Sb mutant HA library,” or the “the Sb mutant vaccine.” When the inventors evaluated the efficacy of this library as a vaccine in mice, they found that, compared to the parental wild-type HA protein, the Sb mutant HA library induced a stronger immune response and disproportionately boosted the immune response against conserved, non-antigenic HA domains (see FIG. 16 for a schematic depiction of these results). As a result, the Sb mutant HA library provides better protection against challenge with both matched and highly drifted influenza strains in mice. Thus, this vaccine has potential for use as a universal flu vaccine.

### Protein Libraries:

In a first aspect, the present invention provides protein libraries (i.e., collections of proteins) comprising variants of an antigenic viral protein. Each variant of the antigenic viral protein comprises a different set of mutations at one or more hypervariable sites.

The terms “protein,” “polypeptide,” and “peptide” are used interchangeably herein to refer to a series of amino acid residues connected by peptide bonds between the alpha-amino and carboxy groups of adjacent residues, forming a polymer of amino acids. Proteins may include modified amino acids. Suitable protein modifications include, but are not limited to, acylation, acetylation, formylation, lipoylation, myristoylation, palmitoylation, alkylation, isoprenylation, prenylation, amidation at C-terminus, glycosylation, glycation, polysialylation, glypiation, and phosphorylation. Proteins may also include amino acid analogs.

As used herein, the term “variants” describes a set of related proteins that comprise at least one mutation relative to each other. A “mutation” is a difference in an amino acid sequence relative to a reference sequence. Mutations include insertions, deletions, and substitutions of an amino acid residue relative to a reference sequence. In some embodiments, the protein library comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, at least 600, at least 700, at least 800, at least 900, at least 1000, at least 1×10⁴, at least 1×10⁵, at least 1×10⁶, at least 1×10⁷, at least 1×10⁸, at least 1×10⁹, at least 1×10¹⁰, or more variants of the antigenic viral protein.

The variants of the antigenic viral protein included in the protein libraries each comprise a different set of mutations at one or more variable or hypervariable sites. A “variable site” or hypervariable site” is position in a viral protein at which the amino acid frequently changes between different strains of a virus. In contrast, a “conserved site” is a position in a viral protein at which the amino acid infrequently changes between different strains of a virus. These variable or hypervariable sites in viruses are known to those of skill in the art and the key distinction is that by generating libraries of proteins or viruses with mutations in these variable sites, the inventors demonstrate that vaccination can push the immune response generated to a vaccine including these libraries to be directed to the more conserved regions of the virus. See FIG. 16.

An “antigenic protein” is a protein that induces an immune response (i.e., the production of antibodies) in the body. The antigenic viral protein on which the protein library is based may be any viral protein that comprises hypervariable sites. Examples of viruses that encode such proteins include, without limitation, influenza virus, human cytomegalovirus (HCMV), respiratory syncytial virus (RSV), severe acute respiratory syndrome coronavirus 2 (SARS-CoV-2), human immunodeficiency virus (HIV), and hepatitis C virus (HCV). For example, influenza comprises hypervariable sites in the protein hemagglutinin¹⁻⁷, HCMV comprises hypervariable sites in the proteins gB, gN, and gO⁸, RSV comprises hypervariable sites in the G protein⁹, SARS-CoV-2 comprises hypervariable sites in the spike protein¹⁰, HIV-1 comprises hypervariable sites in the protein gp120¹¹, and HCV comprises hypervariable sites in the proteins E1 and E2¹². Thus, in some embodiments, the antigenic viral protein is a protein found in a virus selected from influenza, HCMV, RSV, SARS-CoV-2, HIV, and HCV.

In the Examples, the inventors generate a protein library based on the influenza protein hemagglutinin (HA). Thus, in preferred embodiments, the antigenic viral protein is an HA protein. Influenza viruses are enveloped negative-sense single-strand RNA viruses with a segmented genome that encodes a handful of genes. Two of these genes, HA (which facilitates viral entry) and neuraminidase (NA; which facilitates viral release), encode viral glycoproteins that are expressed on the surface of the virion. The HA and NA proteins are the most antigenically variable influenza proteins and are the main targets for protective antibodies generated following influenza infection or vaccination. Circulating viral strains are named for their HA and NA protein subtypes (e.g., H1N1), and yearly vaccines are designed based on the subtypes of the strains most common in the population during a specific year.

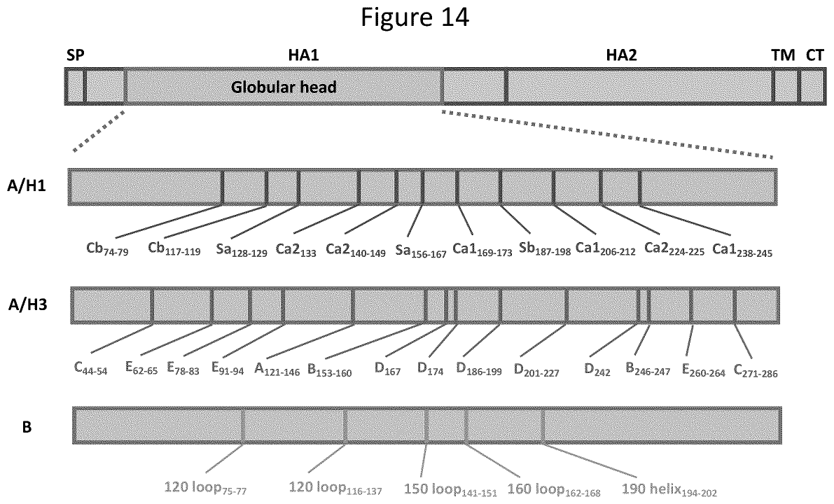

The HA proteins expressed by different subtypes of influenza comprise different hypervariable sites¹⁻⁷. For example, the HA protein expressed by the group one influenza A (e.g., H1) subtype comprises hypervariable residues within the Ca1 site (i.e., amino acids 169-173, 206-212, and 238-245), the Ca2 site (i.e., amino acids 133, 140-149, and 224-225), the Cb site (i.e., amino acids 74-79 and 117-119), the Sa site (i.e., amino acids 128-129 and 156-167), and the Sb site (i.e., amino acids 187-198), whereas the HA protein expressed by the group two influenza A (e.g., H3) subtype comprises hypervariable residues within the A site (i.e., amino acids 121-146), the B site (i.e., amino acids 153-160, 186-199, and 246-247), the C site (i.e., amino acids 44-54 and 271-286), the D site (i.e., amino acids 167, 174, 201-227, and 242), and the E site (i.e., amino acids 62-65, 78-83, 91-94, and 260-264). The HA protein expressed by the influenza B subtype (e.g. the HA of the Victoria or Yamagata lineage) comprises hypervariable residues within the 120 loop (i.e., amino acids 75-77 and 116-137), the 150 loop (i.e., amino acids 141-151), the 160 loop (i.e., amino acids 162-168), and the 190 helix (i.e., amino acids 194-202). The hypervariable regions found in each of these HA proteins are depicted schematically in FIG. 14. Thus, in some embodiments, the antigenic viral protein is influenza A HA 1, influenza A HA 3, or influenza B HA. In some embodiments, the one or more hypervariable sites are in a region of HA 1 selected from Cb, Sa, Ca2, Cal, and Sb, as described above. In other embodiments, the one or more hypervariable sites are in a region of HA 3 selected from A, B, C, D, and E, as described above. In other embodiments, the one or more hypervariable sites are in a region of B HA selected from the 120 loop, 150 loop, 160 loop, and 190 helix, as described above.

In the Examples, the inventors generated a protein library comprising variants of the HA protein from the influenza strain PR8, which is a mouse adapted H1N1 influenza virus. The variants comprise mutations at four positions in the Sb region of HA: Q192, N193, Q196, and E198. Thus, in specific embodiments, the one or more hypervariable sites are selected from the positions Sb 192, Sb 193, Sb 196, and Sb 198.

### Nucleic Acid Libraries:

In a second aspect, the present invention provides nucleic acid libraries (i.e., collections of nucleic acids) encoding the protein libraries disclosed herein. Each nucleic acid in the nucleic acid library encodes one of the variants of the antigenic viral protein of the protein library.

The terms “nucleic acid,” “oligonucleotide,” and “polynucleotide” are used interchangeably to refer a polymer of DNA or RNA. A nucleic acid may be single-stranded or double-stranded and may represent the sense or the antisense strand. A nucleic acid may be synthesized or obtained from a natural source. A nucleic acid may contain natural, non-natural, or altered nucleotides, as well as natural, non-natural, or altered internucleotide linkages (e.g., phosphoroamidate linkages, phosphorothioate linkages).

In some embodiments, the nucleic acids included in the libraries comprise expression constructs, i.e., constructs in which a sequence encoding a protein is operatively linked to a regulatory element that drives its expression in a cell. Specifically, in some embodiments, each nucleic acid in the nucleic acid library further comprises a promoter operably linked to the sequence encoding the variant of the antigenic viral protein.

A “promoter” is a DNA sequence that defines where transcription of a gene or coding sequence begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a coding sequence if the promoter is positioned such that it can affect transcription of the coding sequence.

### Vector Libraries:

In a third aspect, the present invention provides vector libraries (i.e., collections of vectors) comprising a nucleic acid library disclosed herein. Each vector in the vector library comprises one of the nucleic acids of the nucleic acid library.

The term “vector”, as used herein, refers to a nucleic acid that is capable of transporting another nucleic acid to which it is linked. Some vectors are capable of autonomous replication in a host cell into which they are introduced (e.g., bacterial vectors that include a bacterial origin of replication and episomal mammalian vectors). Other vectors can be integrated into the genome of a host cell such that they are replicated along with the host genome (e.g., viral vectors and transposons). Vectors may carry heterogeneous genetic elements that are necessary for propagation of the vector or for expression of an encoded gene product. Vectors may also carry a selectable marker gene, i.e., a gene that confers a selective advantage to a host organism, such as resistance to a drug or chemical, or a detectable marker gene, i.e., a gene that encodes a molecule that creates a detectable signal. Suitable vectors include plasmids (i.e., circular double-stranded DNA molecules) and viral vectors.

As used herein, a “viral vector” is a recombinant viral nucleic acid that has been engineered to express a heterologous protein. Viral vectors include cis-acting elements that drive the expression of the encoded heterologous protein. Suitable viral vectors include, but are not limited to, adenovirus vectors, adeno-associated virus vectors, pox virus vectors (e.g., fowlpox virus vectors), alpha virus vectors, baculoviral vectors, herpes virus vectors, retrovirus vectors (e.g., lentivirus vectors), Modified Vaccinia virus Ankara vectors, Ross River virus vectors, Sindbis virus vectors, Semliki Forest virus vectors, and Venezuelan Equine Encephalitis virus vectors. In a preferred embodiment, the viral vector is a lentiviral vector.

### Virus Libraries:

In a fourth aspect, the present invention provides virus libraries (i.e., collections of viruses) comprising a vector library or nucleic acid library disclosed herein. Each virus in the virus library comprises one of the vectors of the vector library or one of the nucleic acids of the nucleic acid library.

In some embodiments, the virus used in the library is selected from the group consisting of influenza virus, HIV, SARS-CoV-2, HCV, RSV, and CMV.

In some embodiments, the virus is inactivated or attenuated. The term “inactivated” is used to describe a pathogenic virus that has been killed such that it can no longer cause disease. Viruses may be inactivated using heat, chemicals (e.g., formaldehyde, formalin, beta-propiolactone), or radiation. The term “attenuated” is used to describe a pathogenic virus that has been weakened so that it cannot cause disease. Live attenuated viruses are often used as vaccines because they tend to stimulate a stronger and more durable immune response than inactivated viruses. Viruses may be attenuated by serial passaging the virus through a foreign host (e.g., tissue culture, embryonated chicken eggs, live animals). As the virus evolves in the new host, it will gradually lose its efficacy in the original host due to the lack of selection pressure. Viruses may also be attenuated via reverse genetics (e.g., introduction of a mutation that weakens the virus). For example, mutations that cold-adapt the virus (i.e., limit its replication above a particular temperature, thereby limiting the spread of the virus in the respiratory tract) can be introduced.

### Vaccines:

In a fifth aspect, the present invention provides vaccines comprising a protein library, nucleic acid library, vector library or virus library described herein. The vaccine may also include a pharmaceutically acceptable carrier or diluent. The vaccine may also include an adjuvant. As used herein, the term “vaccine” refers to a composition containing an antigen. A vaccine is administered to a subject to stimulate an immune response against said antigen in the subject. The term “antigen” refers to a molecule that can induce a humoral and/or a cellular immune response in a recipient. In the vaccines of the present invention, each of the variants of the antigenic viral protein serve as an antigen. In some embodiments, the vaccine library comprises the library (protein, nucleic acid, vector or virus library) delivered via a nano material (e.g., a nanoparticle or nanofiber).

The term “adjuvant” refers to a compound that enhances a subject's immune response to an antigen when it is co-administered with that antigen. Exemplary adjuvants include, without limitation, aluminum hydroxide, alum, Alhydrogel™ (aluminum trihydrate) or another aluminum-comprising salt, virosomes, nucleic acids comprising CpG motifs such as CpG oligodeoxynucleotides (CpG-ODN), squalene, oils, MF59 (Novartis), LTK63 (Novartis), QS21, saponins, virus-like particles, monomycolyl glycerol (MMG), monophosphoryl-lipid A (MPL)/trehalose dicorynomycolate, toll-like receptor agonists, copolymers such as polyoxypropylene and polyoxyethylene, AblSCO, ISCOM (AbISCO-100), montanide ISA 51, Montanide ISA 720+CpG, etc. or any combination thereof.

### Methods of Inducing an Immune Response:

In a sixth aspect, the present invention provides methods of inducing an immune response against the antigenic viral protein in a subject. The methods comprise: administering to the subject a protein library, nucleic acid library, vector library, virus library or vaccine disclosed herein.

An “immune response” is the reaction of the body to the presence of a foreign substance (i.e., an antigen). The immune response induced by the present methods may comprise a humoral immune response (e.g., a B cell or antibody response), a cell-mediated immune response (e.g., a T cell immune response), or both a humoral and cell-mediated immune response. The immune response can include the production of antibodies against the antigenic viral protein. The immune response of a subject may be evaluated through measurement of antibody titers, neutralizing antibody response, lymphocyte proliferation assays, or by monitoring signs and symptoms after challenge with the corresponding pathogen, such as weight loss, morbidity, or mortality. As used herein, the term “administering” refers to the introduction of a substance into a subject's body. Common methods of administering a vaccine include oral administration, subcutaneous administration, intramuscular administration, intradermal administration, and intranasal administration. The vaccines can be administered as a single dose or in several doses. For example, the vaccines may be administered two or more times separated by 4 hours, 6 hours, 8 hours, 12 hours, a day, two days, three days, four days, one week, two weeks, three weeks, or more.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

In preferred embodiments, the immune response against the variants of the antigenic viral protein is altered as compared to the immune response against the wild-type antigenic viral protein. The term “wild-type antigenic viral protein” is used herein to refer to the version of the antigenic viral protein that is naturally expressed by a virus. For example, in some embodiments, a greater number of antibodies recognizing distinct epitopes of the protein are generated in response to the variants of the antigenic viral protein as compared to in response to the wild-type antigenic viral protein. In some embodiments, a greater proportion of antibodies are generated against conserved sites as compared to hypervariable sites within the antigenic viral protein in response to the variants of the antigenic viral protein as compared to in response to the wild-type antigenic viral protein. In some embodiments, the immune response is protective against a highly drifted strain of the virus.

“Antigenic drift” is a type of genetic variation that occurs in viruses. It arises from the accumulation of mutations in viral genes that encode virus surface proteins that host antibodies recognize (i.e., antigens). A virus is considered “highly drifted” if it has acquired enough mutations relative to a parental virus that it has an altered ability to be efficiently recognized by the immune system.

In the Examples, the inventors demonstrate that their protein library induced a stronger immune response when it was administered in a prime-boost regimen as compared to when it administered in a single dose. Thus, in some embodiments, the protein library, nucleic acid library, vector library, or vaccine is administered in two or more doses. The data demonstrated that the libraries described herein may also be more effective than traditional single strain vaccines when only a single dose of the vaccine was administered, thus in some embodiments the vaccines described herein allow for only a single administration of the vaccine.

### Methods of Generating Vector Libraries:

In a seventh aspect, the present invention provides methods of generating a vector library described herein. The methods comprise: (a) performing saturation mutagenesis on nucleic acids encoding the antigenic viral protein at one or more hypervariable sites of the antigenic viral protein to obtain mutated nucleic acids; (b) cloning the mutated nucleic acids into viral vectors; (c) packaging the viral vectors into viruses; (d) transducing the viral vectors into cells by infecting the cells with the viruses; (e) identifying infected cells that express the antigenic viral protein; and (f) cloning the mutated nucleic acids encoding variants of the antigenic viral protein found in the infected cells into an expression vector to generate the vector library.

“Saturation mutagenesis” is a random mutagenesis technique in which an amino acid at a specific position within a protein is substituted with all possible amino acids. Saturation mutagenesis generates a library comprising 20×(the number of positions mutated) variants. Saturation mutagenesis is commonly achieved via site-directed mutagenesis PCR (i.e., using a set of primers comprising a randomized codon at the position(s) to be mutated) or via artificial gene synthesis (i.e., using a mixture of nucleotides at the position(s) to be mutated). In some embodiments, saturation mutagenesis is performed using the methods described below.

The viral vectors may be packaged into viruses via virus rescue. “Virus rescue” is a technique that is used to produce recombinant viruses. In this technique, each segment of the viral genome is cloned into a viral rescue plasmid in the form of cDNA. Specifically, the viral segment is cloned into a pol I transcription unit that is flanked by a pol II transcription unit in the viral rescue plasmid. Plasmids encoding each segment of the viral genome are transfected into a cell. In the cell, the plasmids are transcribed to produce negative-sense viral RNA from one strand and positive-sense mRNA from the opposite strand, such that all viral RNAs and mRNAs/proteins are expressed and packaged into viral particles (see, e.g., PNAS 99(17):11411-11416, 2002). Alternatively, the mutated nucleic acids may be cloned into a retroviral or other vector using standard cloning techniques known in the art.

As used herein, the term “transducing” refers to the process by which a virus transfers DNA into a cell. Transduction is accomplished by contacting the cell with the virus.

Cells that express the antigenic viral protein can be identified using an antibody that specifically binds the antigenic viral protein, e.g., via an immunoprecipitation assay, magnetic activating cells sorting (MACS), or fluorescence-activated cell sorting (FACS). In some embodiments, the antibody is an antibody that will only bind to the antigenic viral protein when it is properly folded. Cells that express the antigenic viral protein could also be identified using non-antibody peptides, proteins, or nucleic acids (e.g., aptamers) that specifically bind to the antigenic viral protein. Alternatively, cells that express the antigenic viral protein can be identified by fusing the antigenic viral protein to a protein tag or a selectable marker.

### Methods of Performing Saturation Mutagenesis:

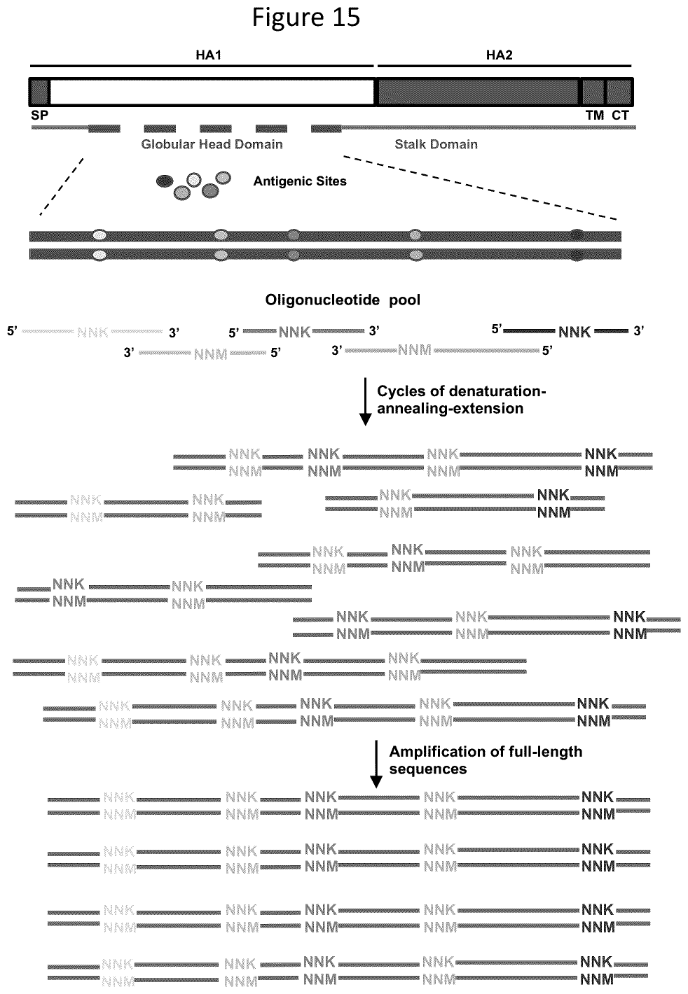

In an eighth aspect, the present invention provides methods of performing saturation mutagenesis on a plurality of target sites within a DNA sequence of interest. These methods are depicted schematically in FIG. 15. The methods comprise: (a) designing a plurality of overlapping primers that are complementary to sequences flanking the target sites and comprise mutations at the target sites; (b) performing a recombination reaction in which the primers are annealed to each other to form a set of recombinant primers that each comprise a different set of mutations at the target sites; and (c) extending the recombinant primers using a DNA polymerase to form full-length sequences that span the target sites; (d) amplifying the full-length sequences to form an amplification reaction product; (e) purifying the amplification reaction product; and (f) cloning the amplification reaction product into an appropriate vector.

As used herein, the term “target site” refers to a site at which saturation mutagenesis is performed. In the methods of the present invention, multiple target sites are simultaneously subjected to saturation mutagenesis using a set of overlapping primers that span the region comprising the target sites.

As used herein, a “primer” is a single-stranded DNA oligonucleotide designed to bind to the sequences flanking a target site via complementary base pairing. DNA polymerases are only capable of adding nucleotides to the 3′-end of an existing nucleic acid. Thus, the binding of a primer to a DNA template strand creates a site from which DNA polymerase can initiate synthesis of a complementary DNA strand in an amplification reaction. Primers can be chemically synthesized or ordered from commercial vendors.

As used herein, the term “complementary” refers to the ability of a nucleic acid molecule to bind to (i.e., hybridize with) another nucleic acid molecule through the formation of hydrogen bonds between specific nucleotides (i.e., A with T or U and G with C), forming a double-stranded molecule. The primers used in the present methods are complementary to the sequences flanking the target sites but comprise non-complementary nucleotides at the target sites so that they can be used to introduce mutations into the protein at the target sites.

In the “recombination reaction” performed in these methods, the overlapping primers anneal to each other, and DNA polymerase is used to fill in the single-stranded regions. In other words, recombination is accomplished via the binding of the homologous portions of the different primers to each other.

A “DNA polymerase” is an enzyme that catalyzes the polymerization of DNA. The polymerase initiates synthesis starting at the 3′-end of the primers annealed to the target sequence, and proceeds in the 5′-direction along the template DNA. Commonly used DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

The term “amplification” refers to a template-dependent process that results in an increase in the concentration of a nucleic acid molecule relative to its initial concentration. A “template-dependent process” is a process in which the sequence of the newly synthesized nucleic acid is dictated by the rules of complementary base pairing. The amplification step of the present methods can be performed using any amplification method known in the art. Exemplary amplification methods include, without limitation, polymerase chain reaction (PCR), loop-mediated isothermal amplification (LAMP), strand displacement amplification (SDA), ligase chain reaction (LCR), and transcription-mediated-amplification (TMA). However, in preferred embodiments, the amplification step is performed using PCR.

As used herein, the term “purifying” refers to the process of separating a desired product from other cellular components and impurities. Suitable methods for purifying an amplification reaction product are well-known in the art and include, but are not limited to, agarose gel purification and commercial PCR clean-up kits.

In some embodiments, the mutations in the primers comprise NNK for forward primers. In some embodiments, the mutations in the primers comprise MNN for reverse primers. In these embodiments, the “N” represents any amino acid.

### REFERENCES

1. Stray S J, Pittman L B. 2012. Subtype- and antigenic site-specific differences in biophysical influences on evolution of influenza virus hemagglutinin. Virol J 9:91.
2. Caton A J, Brownlee G G, Yewdell J W, Gerhard W. 1982. The antigenic structure of the influenza virus A/P R/8/34 hemagglutinin (H1 subtype). Cell 31:417-27.
3. Xu R, Ekiert D C, Krause J C, Hai R, Crowe J E, Jr., Wilson I A. 2010. Structural basis of preexisting immunity to the 2009 H1N1 pandemic influenza virus. Science 328:357-60.
4. Wilson I A, Skehel J J, Wiley D C. 1981. Structure of the haemagglutinin membrane glycoprotein of influenza virus at 3 A resolution. Nature 289:366-73.
5. Wiley D C, Wilson I A, Skehel J J. 1981. Structural identification of the antibody-binding sites of Hong Kong influenza haemagglutinin and their involvement in antigenic variation. Nature 289:373-8.
6. Skehel J J, Stevens D J, Daniels R S, Douglas A R, Knossow M, Wilson I A, Wiley D C. 1984. A carbohydrate side chain on hemagglutinins of Hong Kong influenza viruses inhibits recognition by a monoclonal antibody. Proc Natl Acad Sci USA 81:1779-83.
7. Wang Q, Cheng F, Lu M, Tian X, Ma J. 2008. Crystal structure of unliganded influenza B virus hemagglutinin. J Virol 82:3011-20.
8. Arcangeletti M C, Vasile Simone R, Rodighiero I, De Conto F, Medici M C, Martorana D, Chezzi C, Calderaro A. 2015. Combined genetic variants of human cytomegalovirus envelope glycoproteins as congenital infection markers. Virol J 12:202.
9. Bin Lu, Liu H, Tabor D E, Tovchigrechko A, Qi Y, Ruzin A, Esser M T, Jin H. 2019. Emergence of new antigenic epitopes in the glycoproteins of human respiratory syncytial virus collected from a U S surveillance study, 2015-17. Sci Rep 9(1):3898.
10. Fatihi S, Rathore S, Pathak A K, Gahlot D, Mukerji M, Jatana N, Thukral L. 2021. A rigorous framework for detecting SARS-CoV-2 spike protein mutational ensemble from genomic and structural features. Curr Res Struct Biol 3:290-300.
11. Zolla-Pazner S, Cardozo T. 2010. Structure-function relationships of HIV-1 envelope sequence-variable regions refocus vaccine design. Nat Rev Immunol 10(7):527-35.
12. Stroh L J, Krey T. 2020. HCV Glycoprotein Structure and Implications for B-Cell Vaccine Development. Int J Mol Sci 21(18):6781.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

In the following example, the inventors describe the generation and validation of a viral protein library comprising variants of the antigenic influenza protein hemagglutinin (HA).

### Materials and Methods

Library construction and purification. To make an HA mutant library with mutations in the antigenic site Sb, four positions in the Sb site of PR8 HA, i.e., Q192, N193, Q196, and E198, were chosen for saturation mutagenesis. The sequence of the parental PR8 HA protein was identical to GenBank entry CY083950.1 and had one mutation relative to GenBank entry AF389118. The fragment was cloned into the lentiviral cloning vector pCMV-IRES-GFP version 3 (pCIG3, Addgene, 78264) and lentivirus was packaged. Lentivirus titer on 293T cells was determined by flow cytometry for GFP expression. To sort viable mutants, 293T cells were transduced with lentivirus at MOI of 0.3, and properly folded mutants were isolated by magnetic activating cells sorting (MACS) with monoclonal antibody 6F12 (1) (a kind gift from Dr. Peter Palese) and anti-mouse IgG MicroBeads (Miltenyi, 130048401). The quality of the sorted library was assessed by flow cytometry with 6F12 and allophycocyanin (APC)-conjugated anti-mouse antibody (Invitrogen, A865). The ectodomain of viable mutants was cloned into pDZ mammalian expression vector containing a TEV protease cleavage site and a trimeric motif GCN4pII and a His-tag with Ser-Gly linkers at C-terminus for recombinant protein expression.

Recombinant proteins expression and purification. For soluble proteins used in vaccination and ELISAs, the expression vectors were transiently transfected into Expi293F cells (Gibco, A14527) using ExpiFectamine293 Transfection Kit (Gibco, A14635) following the manufacturer's instructions. Protein was collected on day 3 post transfection and was purified by chromatography on Ni-NTA agarose (Qiagen, 30250) and then was dialyzed against PBS using Slide-A-Lyzer dialysis cassettes (Thermo Scientific).

Deglycosylation of recombinant HA. Recombinant WT HA and Sb mutant HA were denatured and then treated with PNGase F (New England Biolabs, P0704S) following the manufacturer's instructions. The reaction products were visualized by SDS-PAGE.

ELISAs. Ectodomain of PR8 full length HA, PR8 HA head, and PR8 HA stalk were fused to a T4 fibitin foldon, i.e., to avoid measuring false-positive cross reactivity toward GCN4pII used in the vaccine constructs. A His-tag at the C-terminus was used to purify HA protein from 293F cells as described above. H1N1 viruses used in ELISAs were grown in MDCKs (PR8) or eggs (A/WSN/1/1933, A/USSR/92/1977, A/Bayern/07/1995, A/Solomon Islands/03/2006, A/California/04/2009) and were concentrated by sucrose density gradient ultracentrifugation. Monoclonal antibodies used to determine the antigenicity of recombinant proteins including Y8-10C2 (site Sa), Y8-1C1 (site Sb), H37-80 (site Ca), and H2-4C2 (site Cb) were kindly gifted by Dr. Scott Hensley, CR9114 was purchased from Creative Biolabs (PABL-593), PY102 was provide by Dr. Tom Moran (Icahn School of Medicine at Mount Sinai). In IgG isotyping, goat anti-mouse IgG1-HRP, IgG2b-HRP, IgG2c-HRP (SouthernBiotech, 1071-05, 1-91-05, 1078-05) were used as secondary antibodies. In antisera and other monoclonal antibodies detection, goat anti-human IgG-HRP (for CR9114, Invitrogen, A18805) or goat anti-mouse IgG-HRP (for, Invitrogen, A16072) were used as secondary antibodies. Color was developed using tetra-methyl-benzidine (TMB) substrate (Thermo Scientific, 34029), and reactions were stopped with 2M sulfuric acid. Absorbance was measured at 450 nm on a Varioskan Lux microplate reader (Thermo Scientific).

Immunization and challenge. Six- to ten-week-old C57BL/6 female mice were administered 10 μg of recombinant protein in 100 μL of 50% (v/v) mixture of AddaVax (InvivoGen, vac-adx-10) intramuscularly. For infection, mice were administered 40 μL of virus (12,500 PFU of PR8 or 24,000 PFU of Cal09 after prime; 16,000 PFU of PR8 or 24,000 PFU of Cal09 after boost) (2) intranasally after anesthesia with a ketamine-xylazine mixture. Mice were weighed daily and euthanized once their body weight reached <75% of the starting weight as a humane endpoint. Euthanasia was performed via CO₂ as the primary method, and a bilateral thoracotomy was performed as the secondary method. All procedures were approved by the Duke University IACUC.

HAI. Antisera samples were treated with receptor-destroying enzyme (RDE, Denka Seiken) at a 1:4 dilution at 37° C. for 20 h followed by inactivation at 56° C. for 30 min and further dilution to 1:10 with PBS. Samples were 2-fold serially diluted in v-bottom microtiter plates. Virus adjusted to 4 HA units in 25 μL was added to each well. The plates were incubated at room temperature for 15 min followed by the addition of a 50 μL of 0.5% chicken (for PR8) or turkey (for Cal09) erythrocytes (Lampire Biologicals). The reaction mixture was settled for 30 min at room temperature. Wells were examined visually for inhibition of HA. HAI titers were the reciprocal of the highest dilution of serum that completely inhibited HA.

Micro neutralization. Antisera was serial diluted in 96-well microplates. Virus suspension (1500 PFU of PR8 or 900 PFU of Cal09) was added to each well, and the plates were incubated at 37° C. for 1 h. Following incubation, 1.5×10⁴ suspended MDCK cells were added to each well, and the microplates were incubated at 37° C. in an incubator with 5% CO₂ for 20 h. Cells were then fixed using 4% PFA/PBS and stained for HA protein with CR9114 and goat anti-human IgG-HRP. Color was developed by TMB, and absorbance was measured as described above. IC50 values were calculated from neutralization curved using a four-parameter nonlinear regression model.

ADCC reporter assay. ADCC reporter assay was performed using Mouse FcγRIV ADCC Bioassay following the manufacturer's instructions (Promega, M1215). Briefly, 96-well white tissue culture plates (Perkin Elmer, 6005680) were seeded with 1.25×10⁴ MDCK cells in 100 L per well. After 16-20 hours of incubation, cells were infected with 5 MOI of virus and incubated at 37° C. for 20 hours. Then the medium was replaced with 25 μL per well of assay buffer (RPMI 1640 with 4% (V/V) low IgG FBS). Then 25 μL of serial dilutions of samples was added to each well and incubated at 37° C. for 30 min. Then mouse FcγRIV effector cells at a concentration of 7.5×10⁴ in 25 μL were added to each well and incubated at 37° C. for 6-10 hours. A volume of 75 μL of Bio-Glo reagent was added to each well and luciferase was measured by a EnSpire 2300 Multilabel Reader (Perkin Elmer).

Lung histology. Mouse lungs tissues were fixed in 4% PFA/PBS at 4° C. for more than 48 h. Samples were embedded in paraffin and sectioned after dehydration and wax immersion, slides were stained with H&E and then were scanned, respectively, by HistoWiz.

Plaque assay. Viral titers in lungs were determined using a standard plaque assay protocol on MDCK cells. Virus was serially diluted, and was incubated with the cells for 1 h at 37° C. Then virus was removed and a 1% agar overlay containing TPCK-trypsin was applied. After incubation at 37° C. for 48-72 h, cells were fixed in 4% PFA for 3 h. Polyclonal PR8 virus antibody from WT PR8- or WT Cal09-infected mice and goat anti-mouse IgG-HRP antibody were used to stain plaques. Plaques were visualized by TrueBlue peroxidase substrate (SeraCare, 5510-0030) and were counted.

HA binding custom multiplex assay. Soluble ectodomain of HA of the influenza strains used in the assay was cloned into pFastBac expression constructs containing a T4-fibitin trimerization domain and His-tag as described previously (3). Recombinant protein was purified from sf9 cells by GenScript. Bovine serum albumen (BSA; Sigma—negative control) or rHA were conjugated to magnetic microspheres (Luminex Corp) by carbodiimide coupling using Luminex's recommended protocol. rHA- and BSA-coated microspheres (1500 of each) were mixed with samples diluted 1:100 in PBS, 1% BSA pH7.4 (assay buffer; Life Technologies), incubated for 1 h at room temperature on an orbital shaker and then washed in assay buffer. Microspheres were incubated with 4 g/mL phycoerythrin (PE)-polyclonal goat anti-mouse Ig (Southern Biotech, 1036-09) for 30 min, washed in assay buffer, and the PE median fluorescence intensity (MFI) of each microsphere population was measured using a Bio-Plex 200 analyzer (BioRad). Data were analyzed using Bio-Plex Manager v. 6.2 and are reported as background-corrected MFI from duplicate wells.

Statistics. Data were analyzed using Prism software (GraphPad). Values below the limit of detection were assigned a value of one-half the LOD (LOD/2) in subsequent analyses. Unless otherwise noted, significance was determined using unpaired Student's t test or one-way analysis of variance (ANOVA) followed by Tukey's post hoc analysis. Body weight changes and survival after challenge were analyzed by two-way ANOVA followed by Sidak's multiple-comparison test or a log rank (Mantel-Cox) test, respectively. Asterisks in all figures indicate significance (*, P<0.05; **, P<0.001).

### Results:

### Development of an Antigenically Complex Purified HA Subunit Vaccine

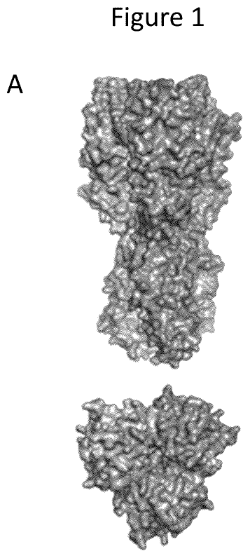

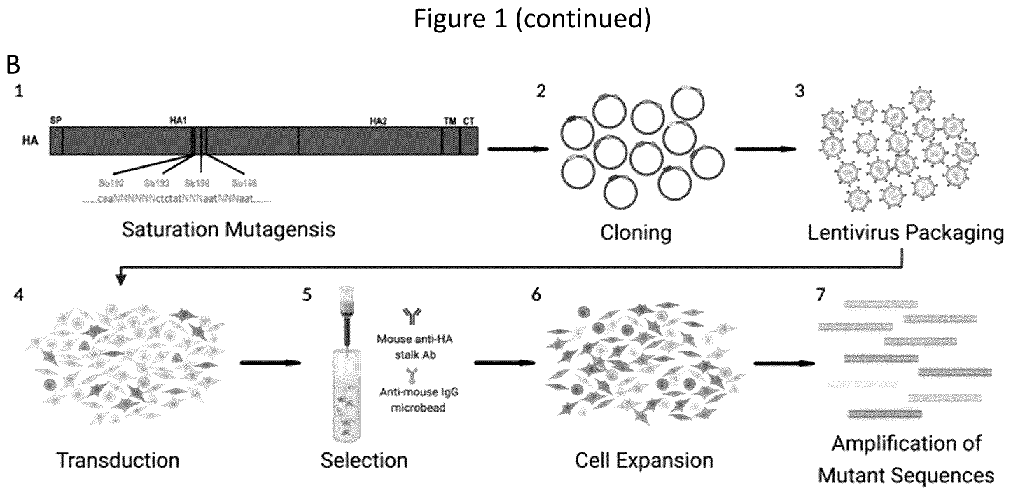

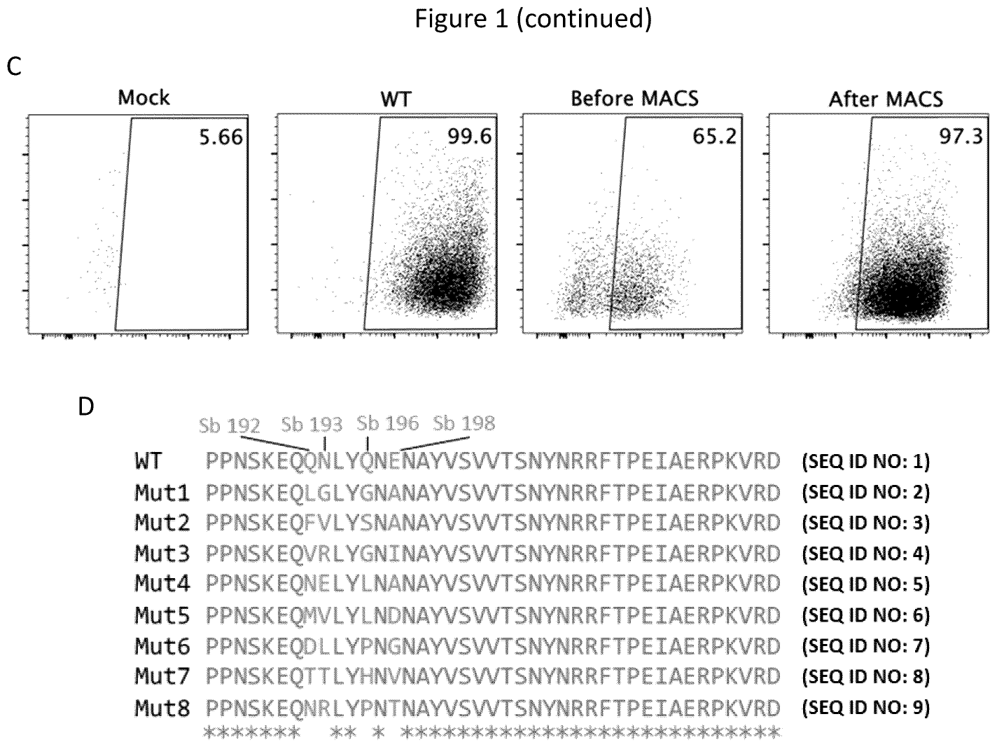

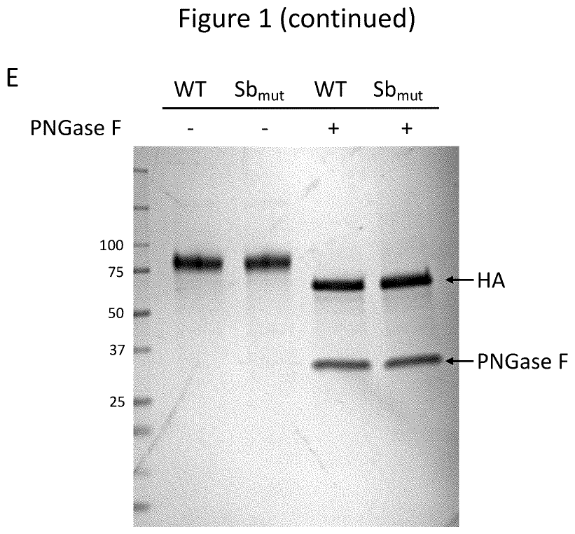

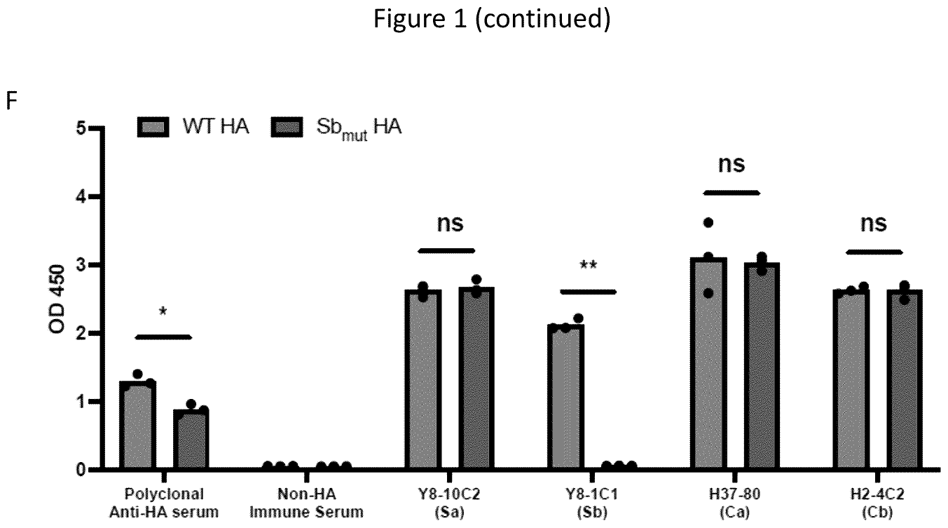

To elicit a more broadly cross-protective immune response against influenza viruses after vaccination, we wanted to alter the ratio of antibodies raised against variable and conserved domains in hemagglutinin (HA). We thus developed an antigenically complex mixture of HA proteins such that the immune response against the protein “population” would be altered relative to vaccination with a standard, homogenous HA. Specifically, we hoped to change the relative “molarity” of antigenic and conserved sites so that the immune system would be more likely to repeatedly encounter conserved domain antigens. We selected the Sb antigenic site of the HA protein from the influenza strain A/Puerto Rico/9/1934 (PR8) H1, which is known to be a dominant antigenic site. Using DNA oligos with randomized codons, we generated a complex mixture of about 10⁶ HA genes (1.75×10⁵ cells were present after sorting; 5×10⁵ pCIG3_PR8 Sbmut HA colonies were present after electroporation) (FIG. 1A,B). We expected that a subset of the mutants would not be expressed or folded correctly. Thus, we delivered the mutant HA proteins via lentivirus vector at low MOI and performed magnetic bead-based sorting with a conformation specific HA stalk antibody (6F12) to purify only HA trimers that folded and trafficked appropriately (FIG. 1C). After cell purification, we amplified the mutant HA genes and cloned the soluble ectodomains into a protein expression vector.

To test our post-purification diversity, we picked individual clones from the purified HA mutant gene library and performed Sanger sequencing. As expected, we found that each clone tested represented a distinct Sb mutant (FIG. 1D). To understand how the Sb mutant HA protein mixture would express relative to the parental HA, we transfected plasmids into 293F suspension cells and purified His-tagged HA ectodomains. SDS-PAGE analysis of the purified proteins revealed bands of similar purity and size, and treatment with PNGase F revealed they were similarly glycosylated (FIG. 1E). Finally, we validated our Sb mutant HA protein mixture by performing ELISA with a panel of antigenic site monoclonal antibodies. While antibodies against the other antigenic sites (Sa, Ca, Cb) bound both the WT and Sb mutant proteins equally, the Sb reactive monoclonal antibody failed to bind the Sb mutant HA library (FIG. 1F). Thus, our approach was successful in generating a recombinant Sb mutant HA protein mixture.

Vaccination with the Parental or Sb Antigenic HA Mixture Elicits Differential Antibody Responses

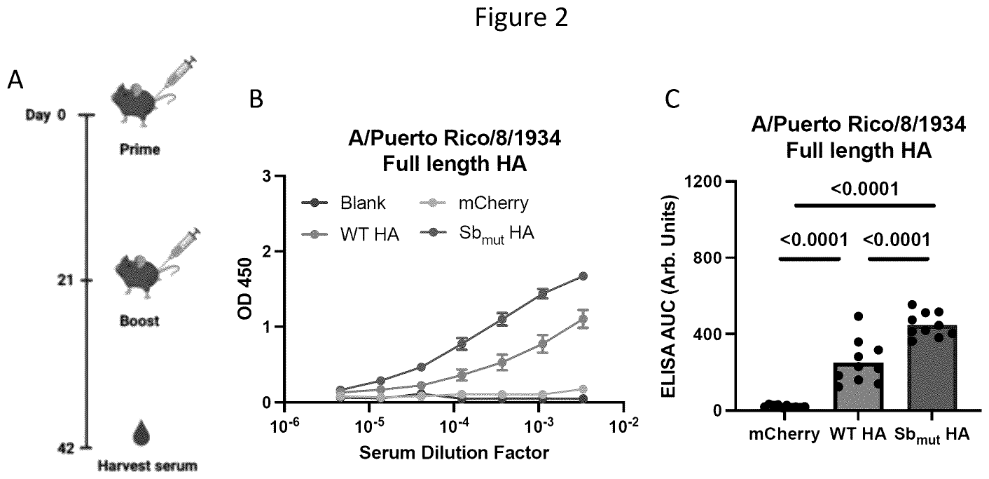

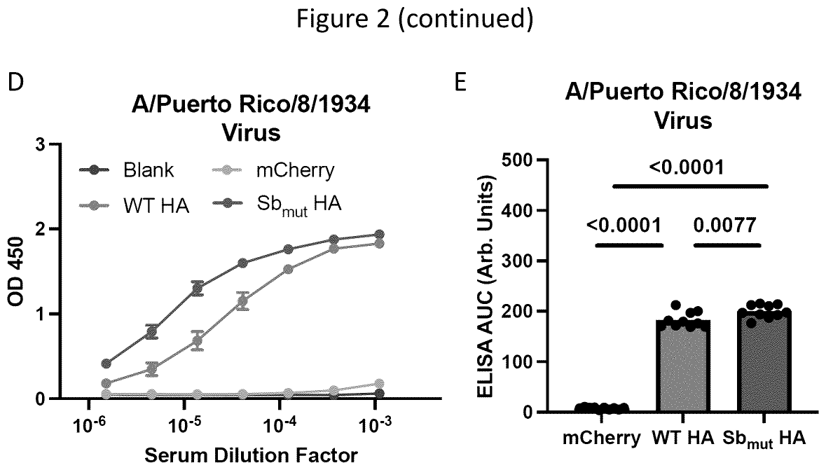

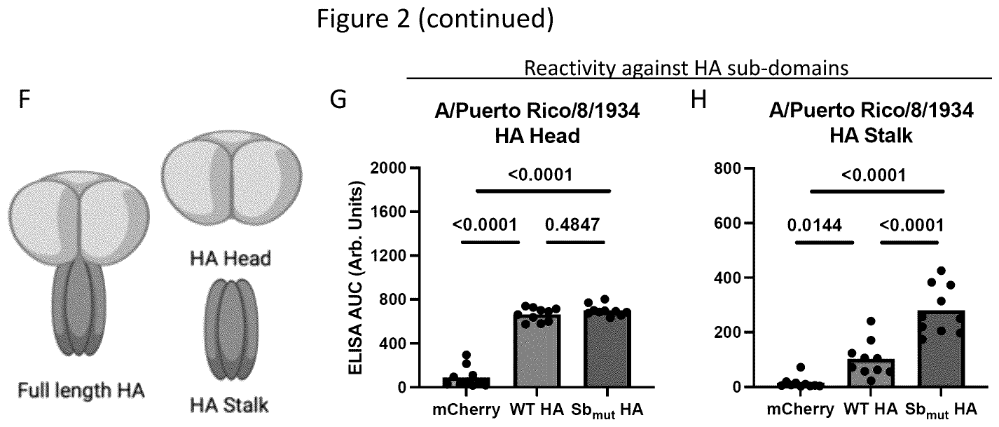

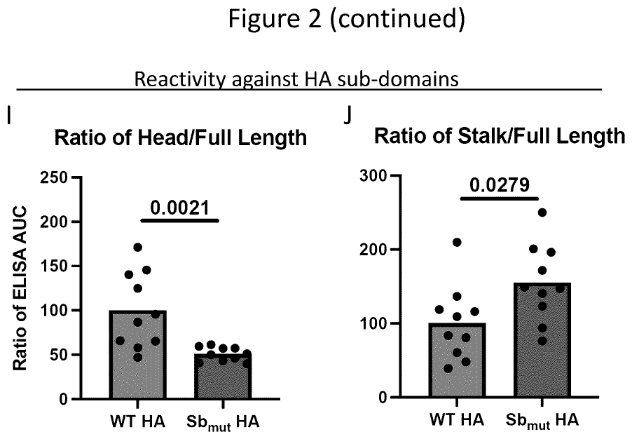

We were next interested in understanding how the immune system would respond to the Sb mutant HA protein mixture relative to homogenous parental HA. We therefore vaccinated mice with WT HA or the Sb mutant HA protein mixture (adjuvanted with Addavax) with a prime-boost regimen 21-days apart (FIG. 2A). Post-vaccination sera was collected and ELISA was performed against the parental full-length purified HA protein. Unexpectedly, the Sb mutant vaccinated sera showed significantly higher binding to full-length HA, indicating that the Sb mutant HA protein mixture was inherently more immunogenic compared to the parental protein (FIG. 2B,C). To evaluate binding to an authentic, virion-displayed HA protein, we repeated the ELISA assay on plates coated with purified PR8 virions. Again, we observed higher sera reactivity with the Sb mutant vaccinated sera (FIG. 2D,E).

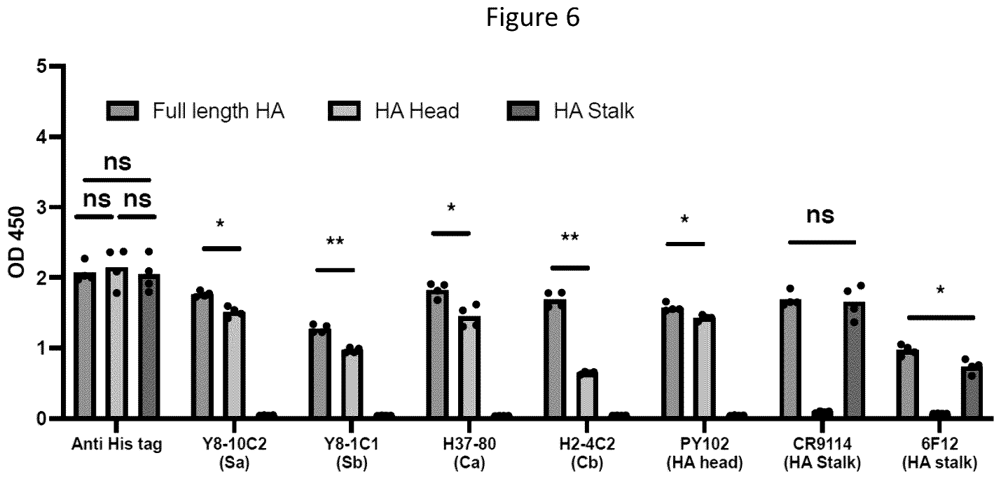

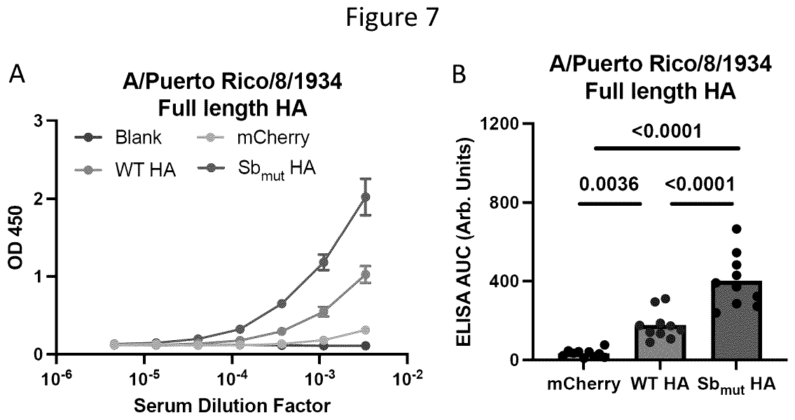

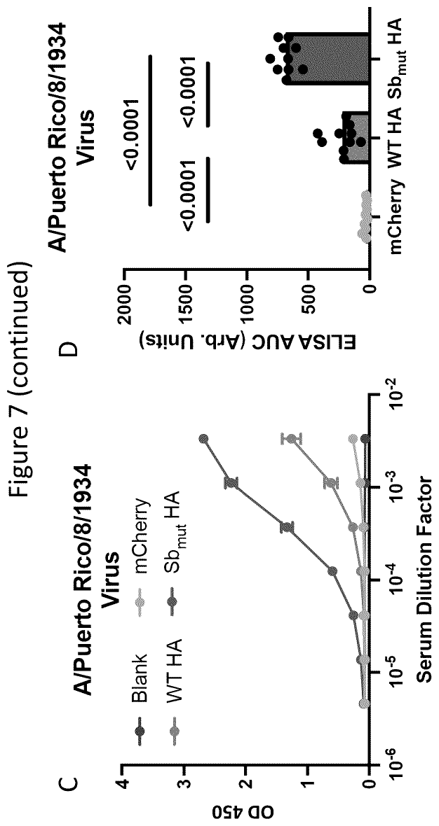

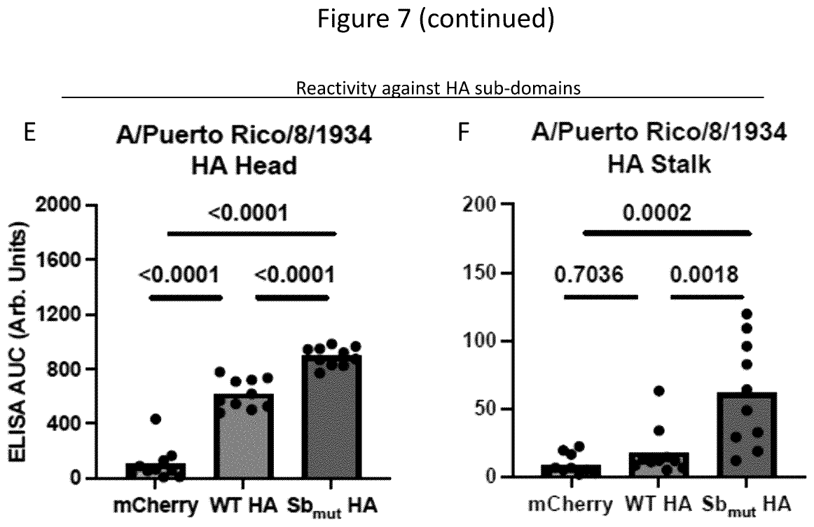

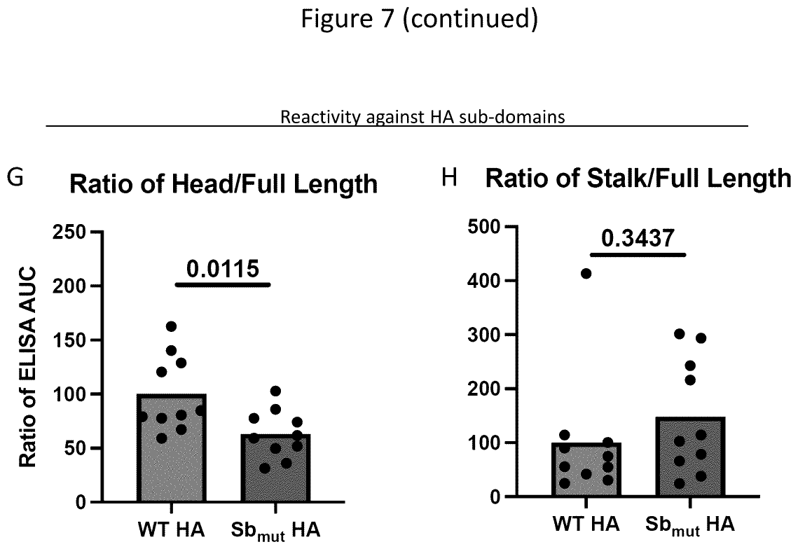

While we observed a boost in overall antibody reactivity, we wanted to evaluate if we were successful in our original goal of shifting antibody responses against conserved or variable domains. We therefore cloned and purified trimerized soluble PR8 HA head and stalk domains (FIG. 6) to measure antibody responses broadly against the generally variable and conserved domains, respectively (FIG. 2F). In contrast to the full-length HA results, sera binding against the HA head domain, while well above background, was not different between the two vaccines (FIG. 2G). The response to the stalk domain, however, was much higher after the Sb mutant vaccination (FIG. 2H). Finally, by normalizing the responses to each HA domain against the full-length HA protein to account for the increased immunogenicity of the Sb mutant vaccine, we observed that relative amounts of antibodies against the variable head domain were reduced while antibodies against the more conserved stalk domain were increased (FIG. 2I,J). Analogous analyses with sera derived from a prime-only vaccination regimen showed similar trends (FIG. 7). Thus, vaccination with the Sb mutant population not only elicits a stronger overall response, but also redirects that response to more conserved HA domains, likely by decreasing reactivity against the Sb antigenic site.

Sb Antigenic Mutant Elicited Antibodies are Functional and Prevent Disease after Challenge

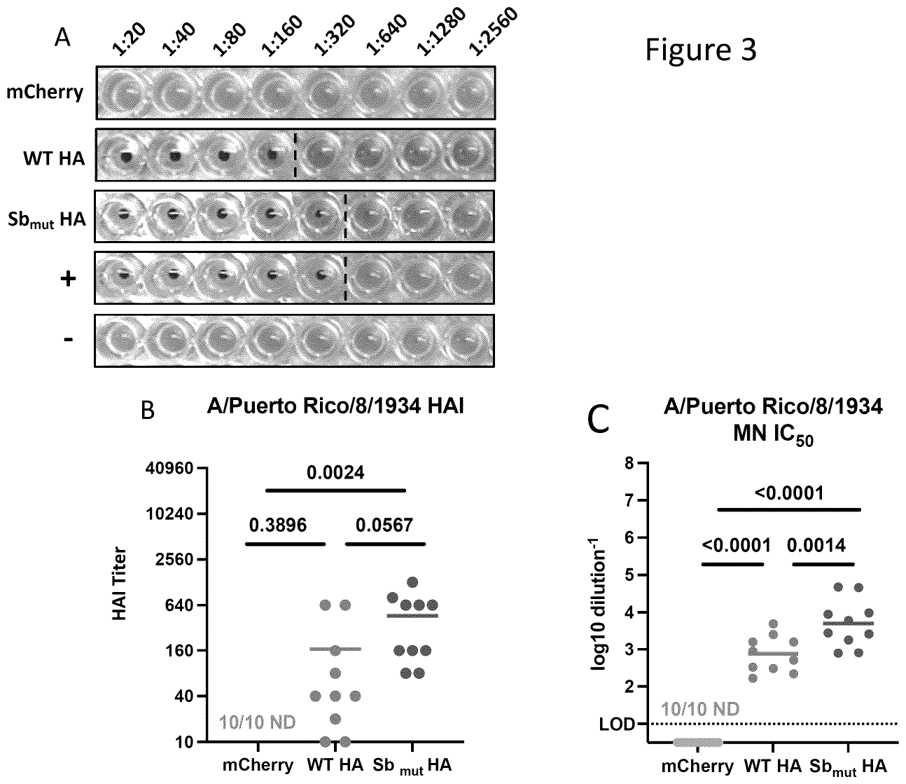

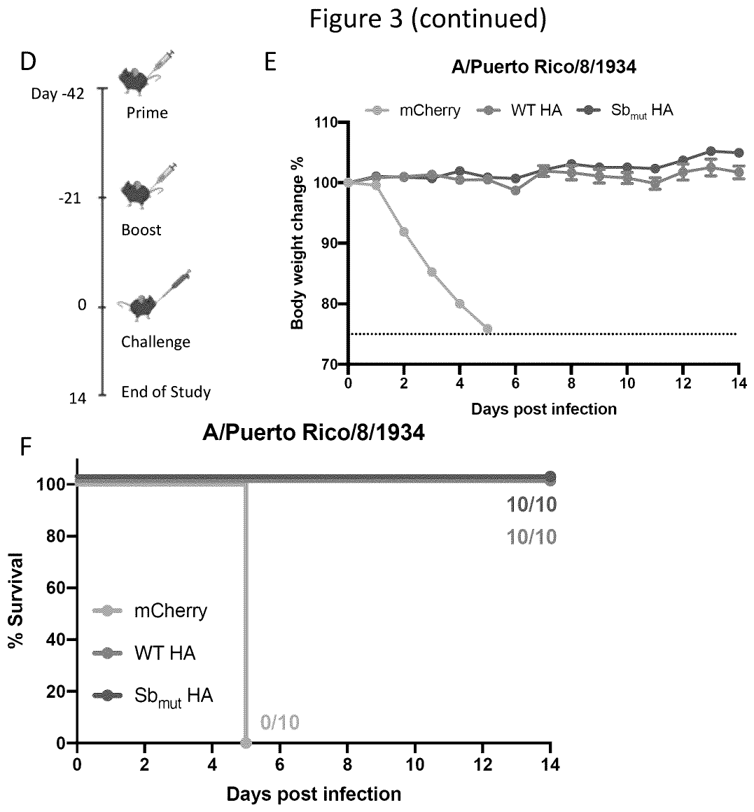

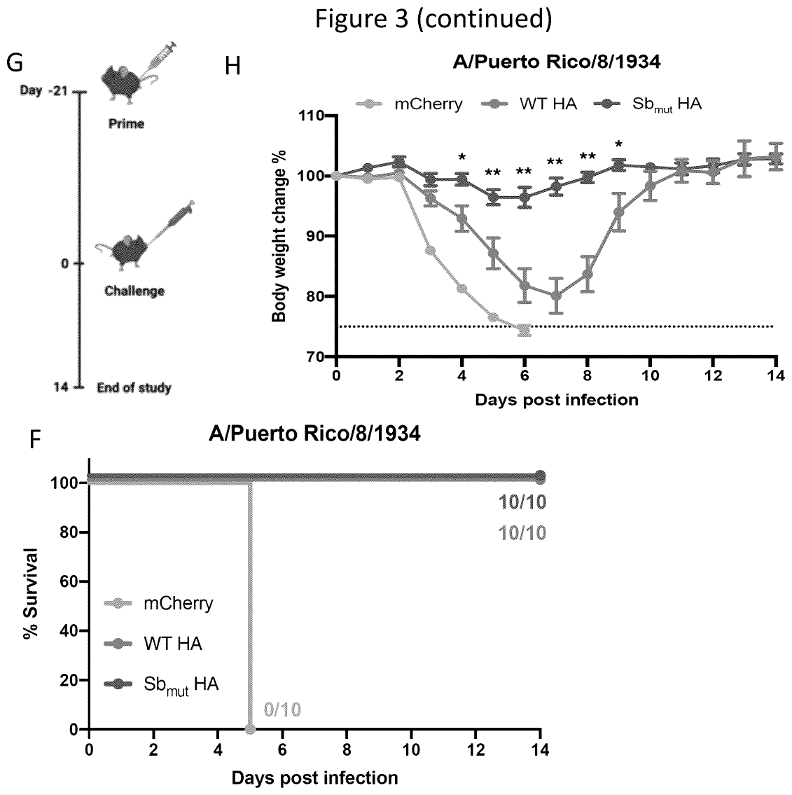

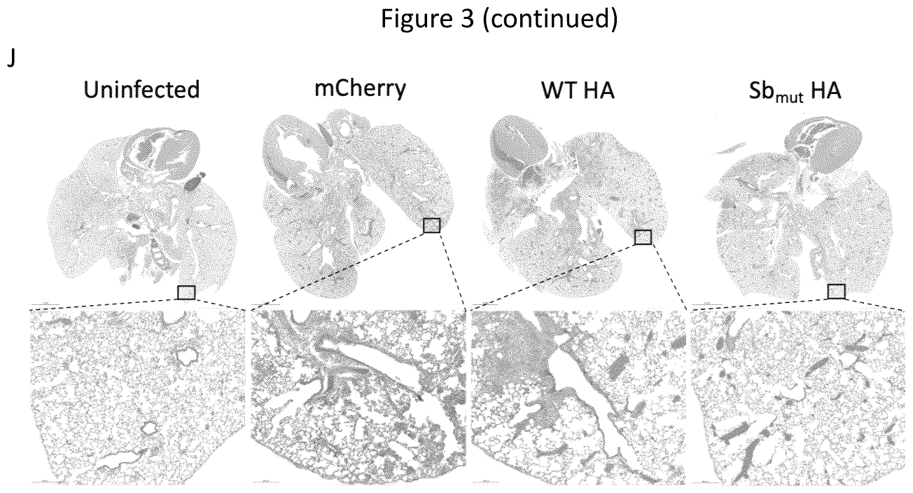

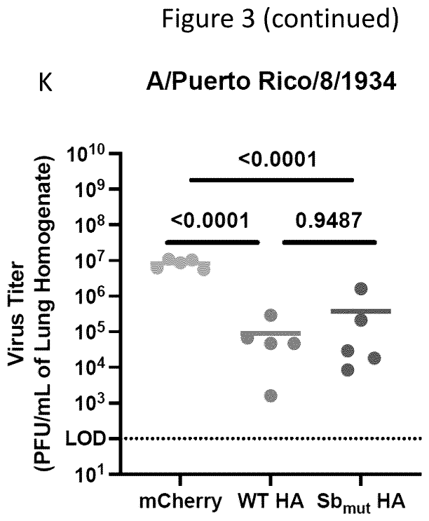

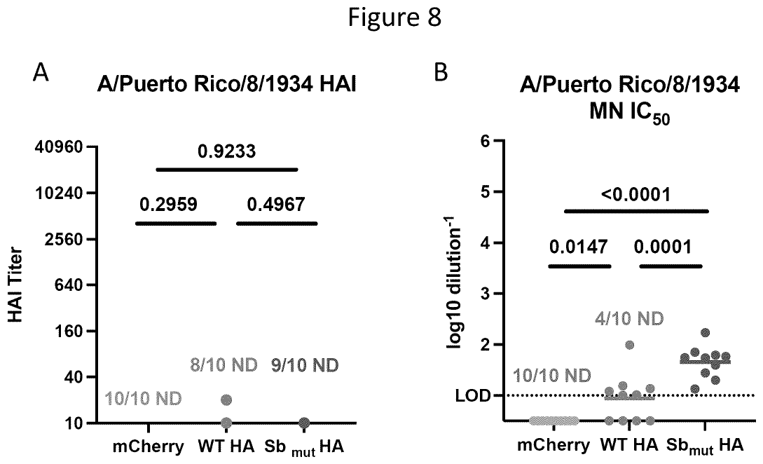

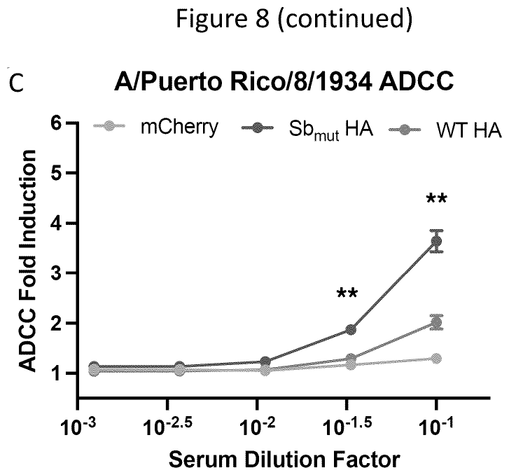

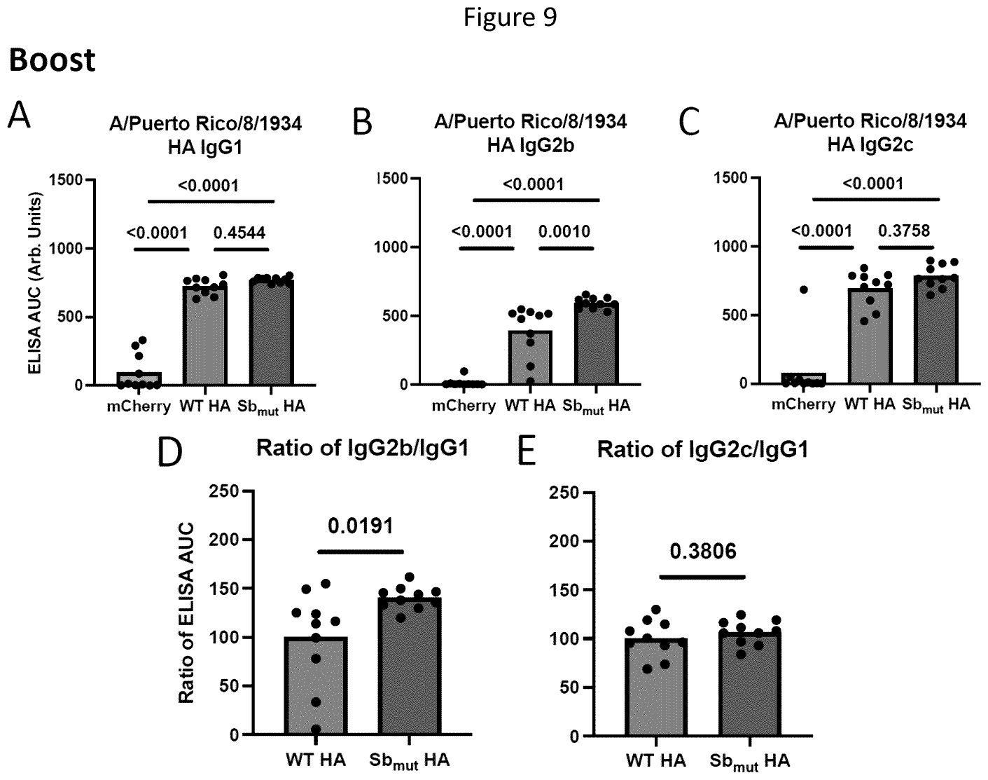

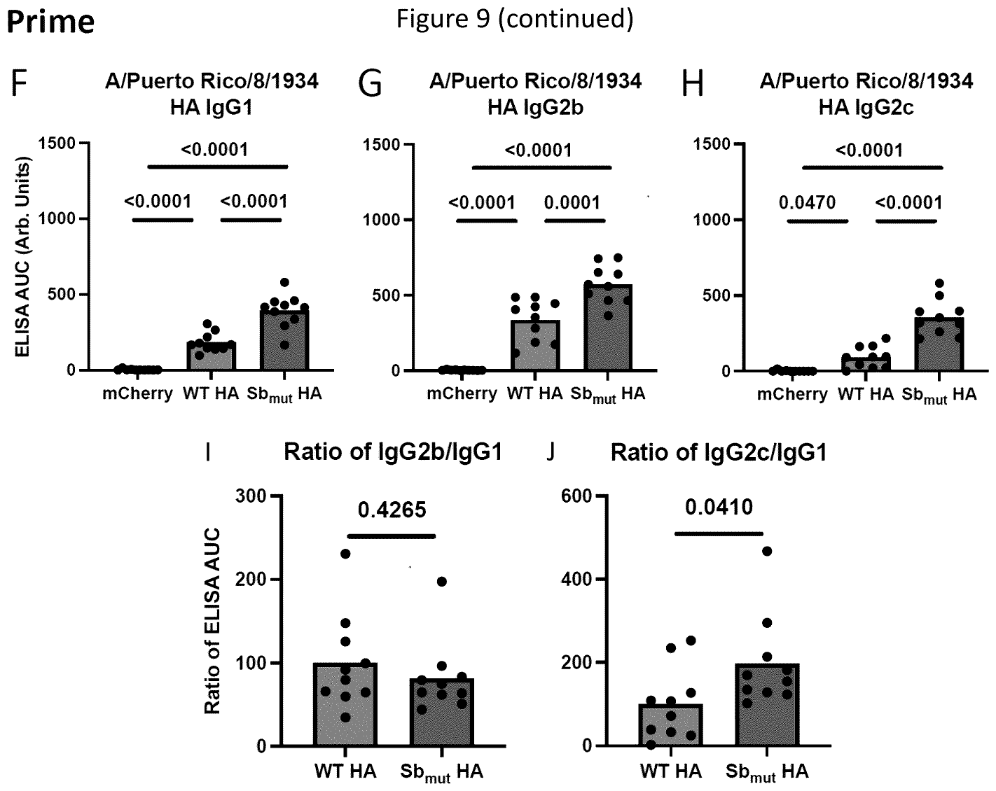

Because in vitro binding does not necessarily correlate with antibody functionality, we next tested our post-vaccination serum in both hemagglutinin inhibition (HAI) and microneutralization (MN) assays. Consistent with higher anti-HA antibodies in the Sb mutant vaccination group, both HAI and MN activities were significantly increased relative to parental HA vaccination (FIG. 3A-C). While prime alone did not elicit a measurable HAI titer, MN showed the same trends as the prime/boost regimen (FIG. 8). Additionally, and because stalk directed antibodies do not frequently have activity in those assays, we measured the ability of the post-vaccination serum to promoter antigen-dependent cellular cytotoxicity (ADCC), a known function of non-neutralizing antibodies. Prime alone vaccination with the Sb mutant HA protein mixture elicited significantly higher signal (FIG. 8). Finally, and consistent with the increased neutralizing and non-neutralizing activities of the serum, we could generally detect higher levels of IgG isotypes associated with those functions (IgG1, IgG2b, and IgG2c, respectively) elicited by the Sb mutant vaccine relative to parental HA after both prime and boost (FIG. 9).

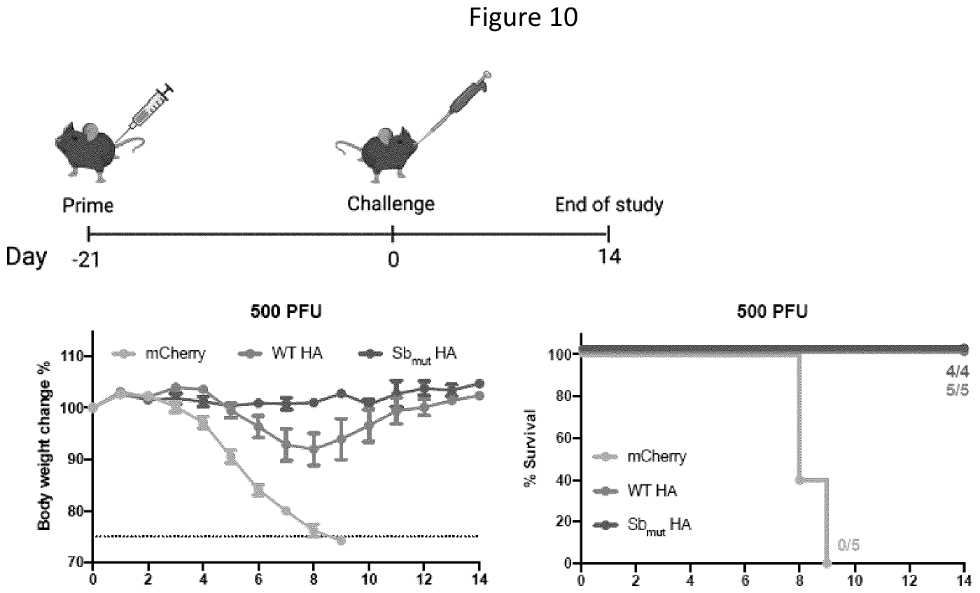

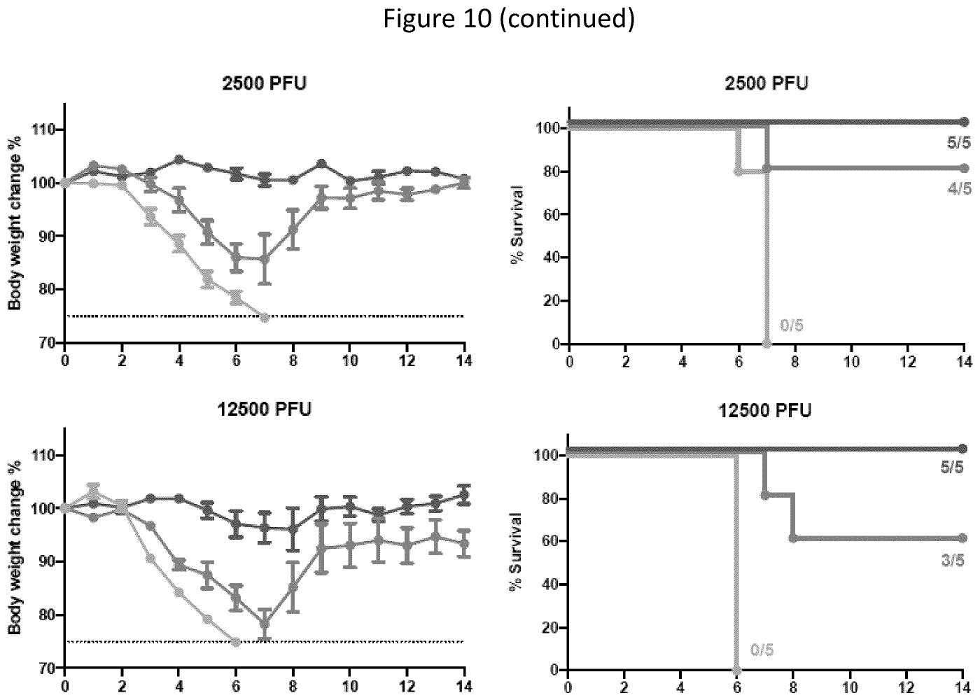

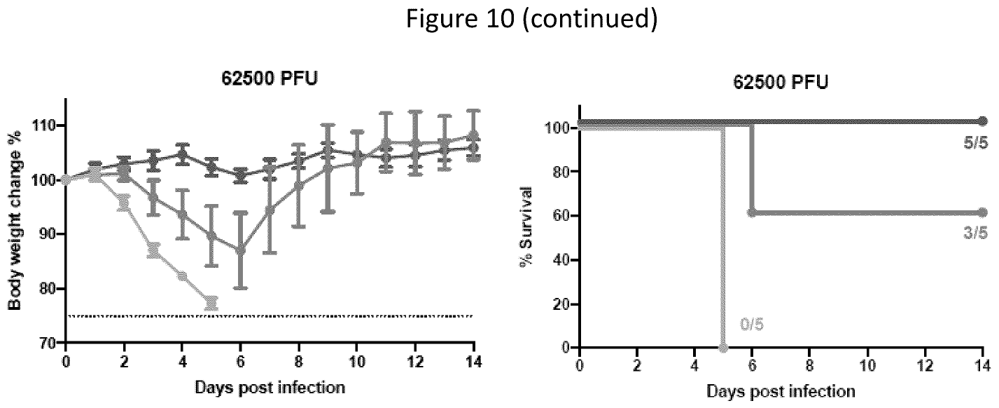

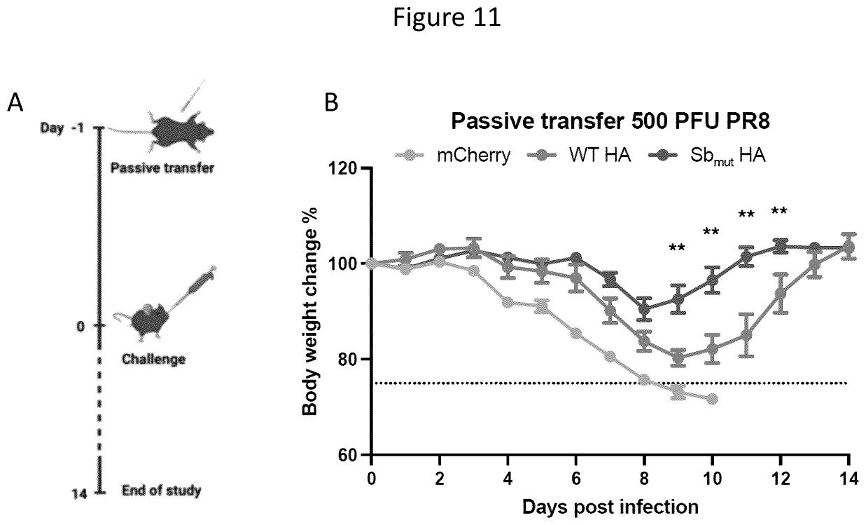

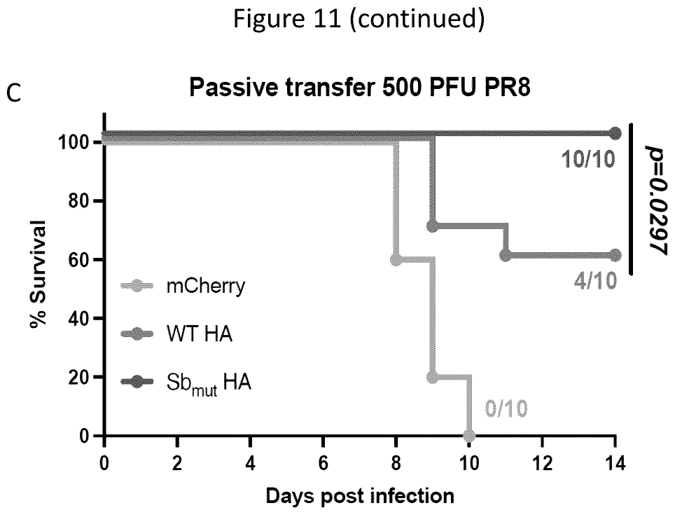

Next, we repeated our vaccination and challenged with a lethal dose of the homologous vaccine virus, PR8. After prime/boost (and with a trimerized mCherry protein as an irrelevant protein control), we observed complete protection from both morbidity and mortality from both vaccines, even at a high challenge dose (FIG. 3D-F). We therefore repeated the challenge in prime-only animals which had similar trends as the prime-boost group but a lower magnitude of responses (FIGS. 7-9). In this case, we could see a significant improvement in both preventing morbidity and allowing survival from challenge in the Sb mutant vaccine (FIG. 3G-I). This trend held true across a range of doses tested, including up to 1250× the LD50 (FIG. 10). Further, we could demonstrate that this protection was at least partially antibody mediated as passive serum transfer experiments showed enhanced protection from the Sb mutant vaccine serum (FIG. 11). Evaluation of lung morphology and immune infiltration during challenge revealed that both vaccines significantly reduced obvious virally induced disease markers, likely predominantly by reducing viral titer (FIG. 3J-K). Interestingly, while both vaccines provided some benefit, the lung morphology was improved in the Sb mutant vaccine group without a corresponding decrease in viral titer. These data suggest that the mechanisms of protection of the Sb mutant vaccine may differ from vaccination with parental HA.

### The Sb Antigenic Mutant Vaccine Elicits Broad Heterologous, Homosubtypic Reactive Antibody Responses

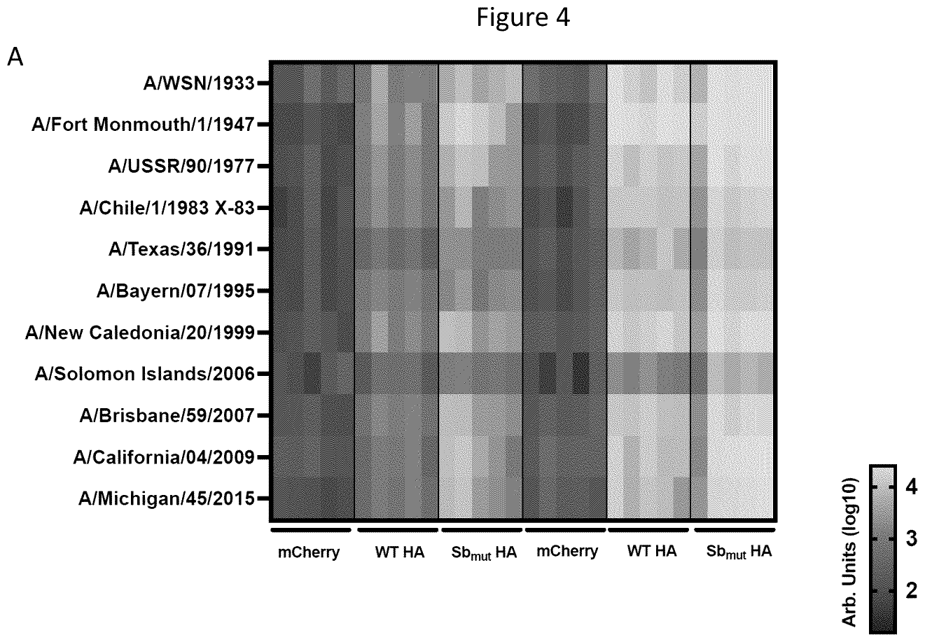

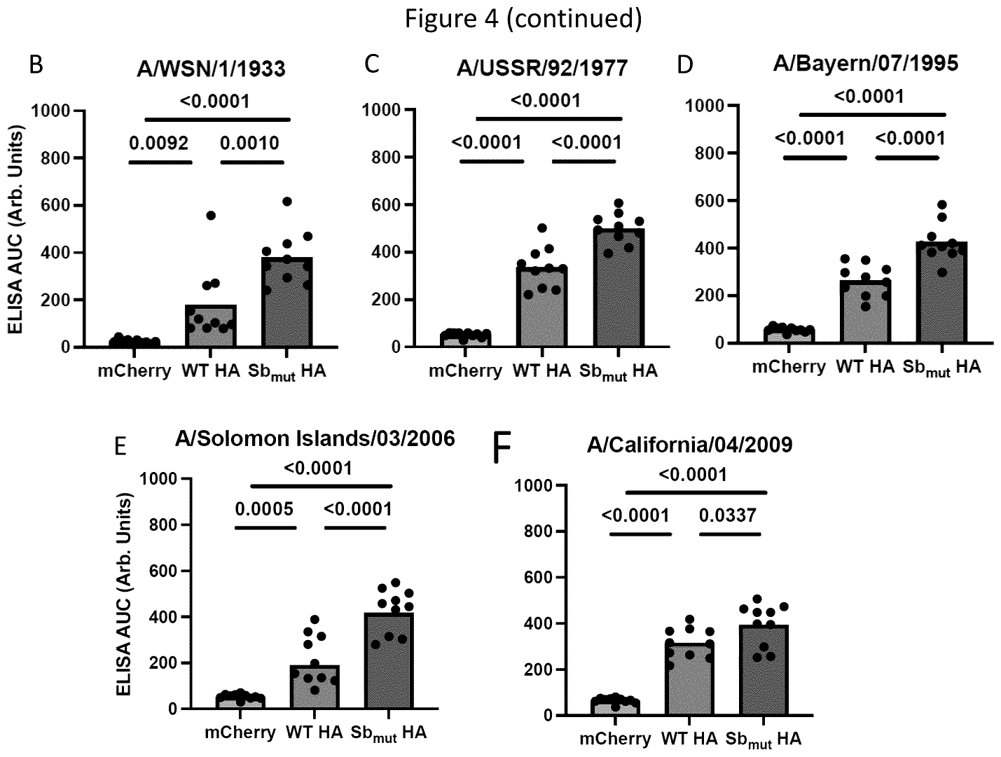

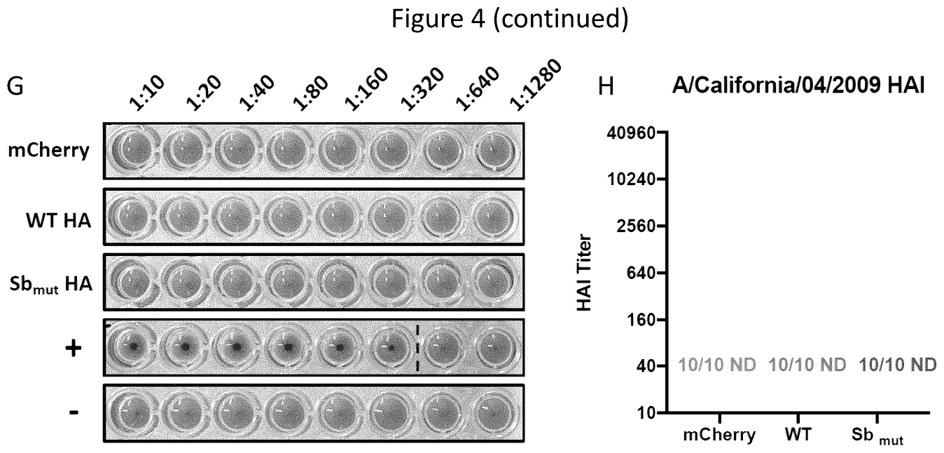

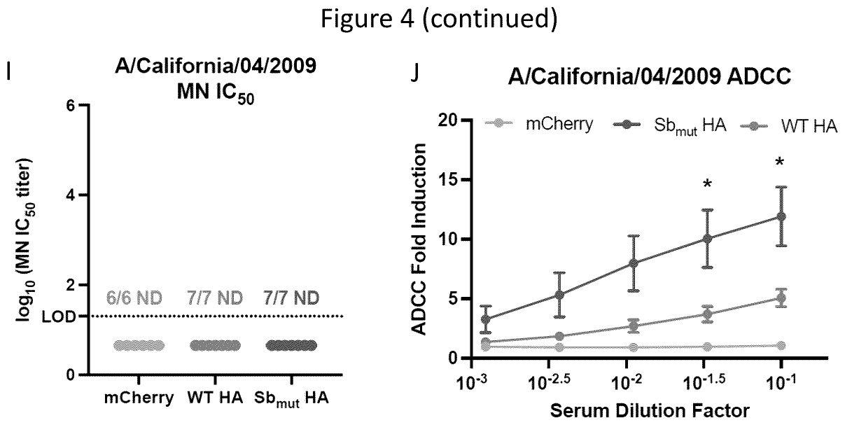

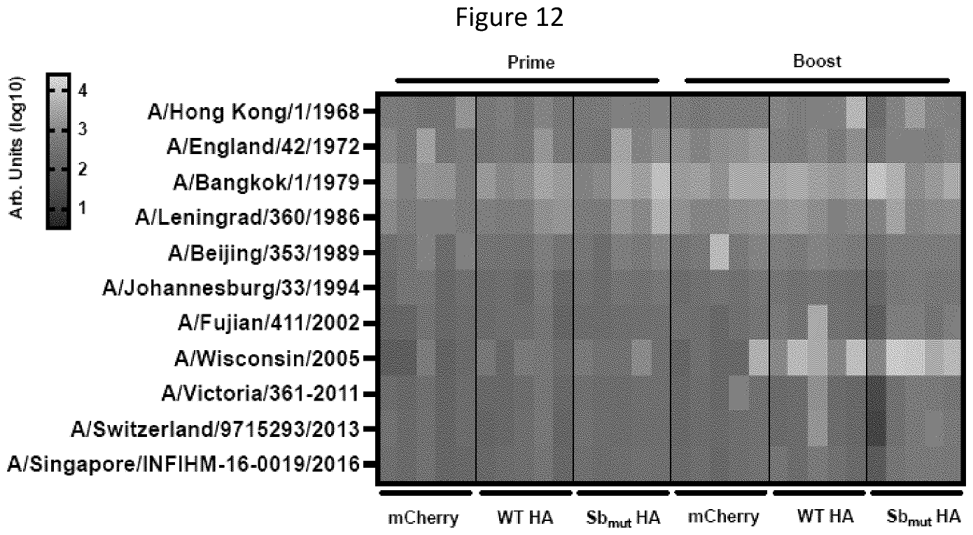

Next, we were interested in understanding if the increased reactivity against more conserved HA domains like the stalk would manifest as increased binding to heterologous HA proteins. Using a Luminex-based purified HA assay, we could detect increased responses against H1 HAs from 1933-2015 in the Sb mutant post-vaccination serum relative to the parental HA vaccine group (FIG. 4A). We could not, however, detect meaningful reactivity to heterologous, cross-group H3 HAs, suggesting the breadth of the responses are likely restricted to either homosubtypic and/or potentially group 1 HA proteins (FIG. 12). To validate the Luminex data, we performed ELISA assays against authentic virions from A/WSN/1/1933, A/USSR/92/1977, A/Bayern/07/1995, A/Solomon Islands/03/2006, and A/California/04/2009. In agreement with the other assay, in all cases, the Sb mutant vaccine serum displayed higher reactivity relative to the parental HA group (FIG. 4B-F).

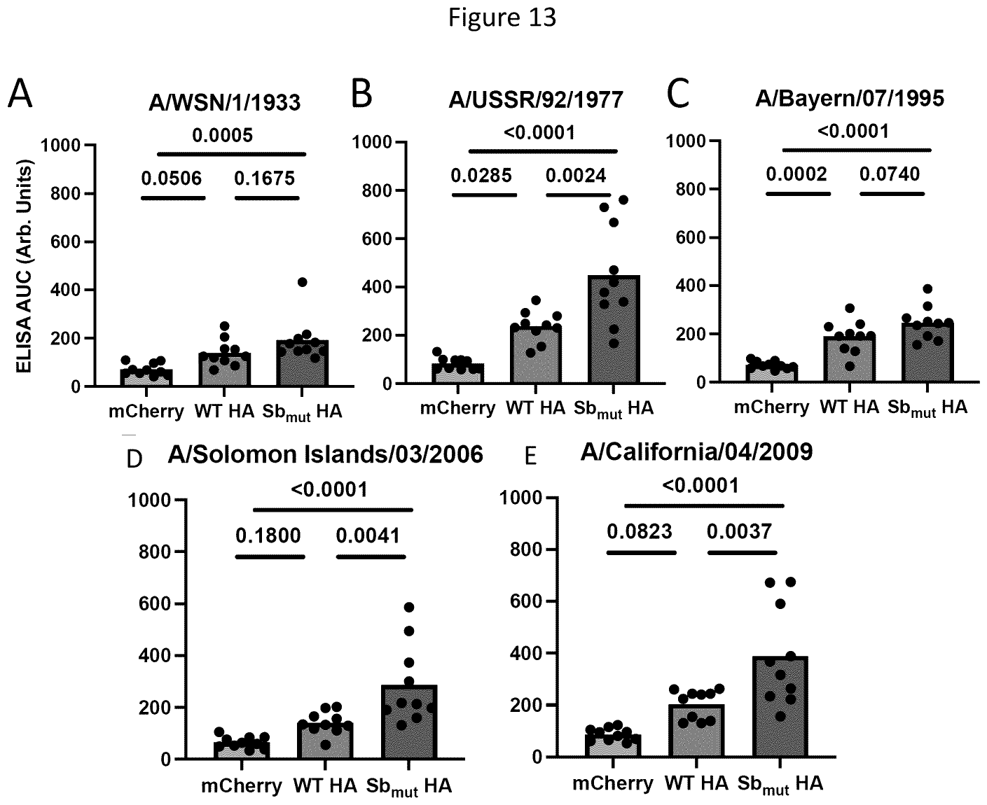

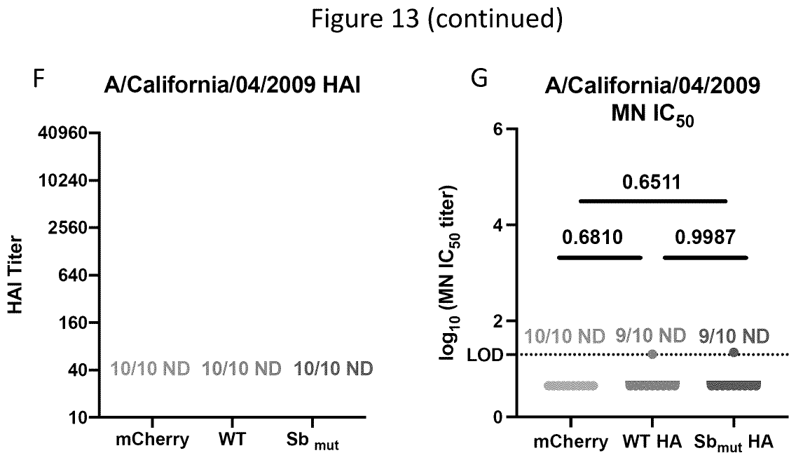

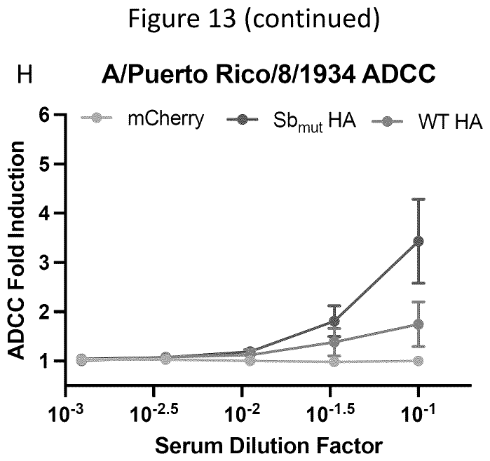

Since we had observed improved reactivity against drifted H1 HAs, we decided to test antibody functionality with the highly drifted, post-pandemic A/California/04/2009 isolate. Consistent with significant divergence in the antigenic sites between the two viruses, we could not detect any meaningful HAI or MN activity in any of the treatment groups (FIG. 4G-I). We could, however, detect significantly more ADCC activity in the Sb mutant vaccine group, which we hypothesize is likely due to higher abundance of cross-reactive but non-neutralizing antibodies elicited by the Sb mutant vaccine (FIG. 4J). Post-vaccination serum from prime-only vaccine groups again showed similar trends as the prime-boost vaccination groups (FIG. 13).

Vaccination of Mice with the Sb Antigenic Mutant Vaccine Leads to Improved Protection from Heterologous H1N1 Challenge

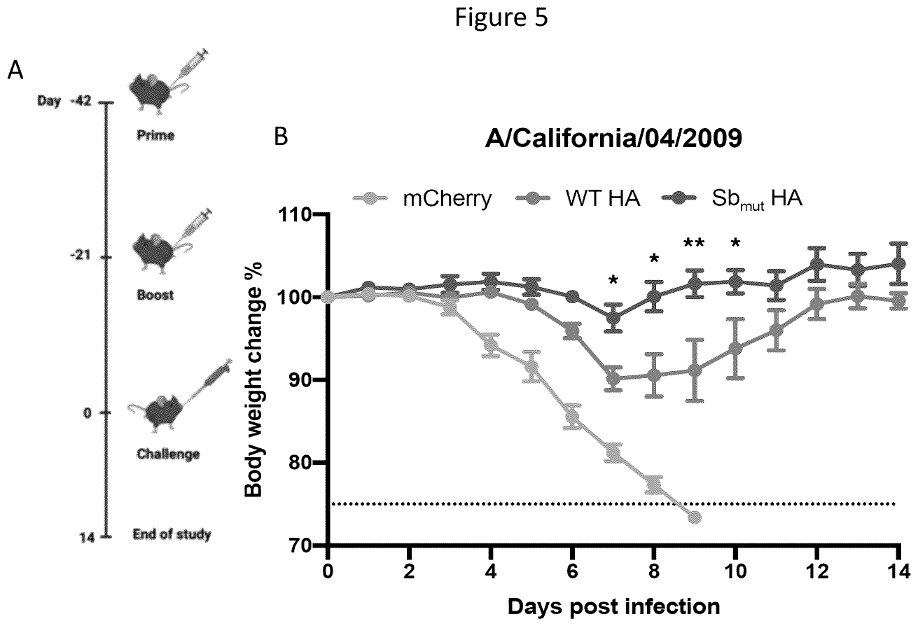

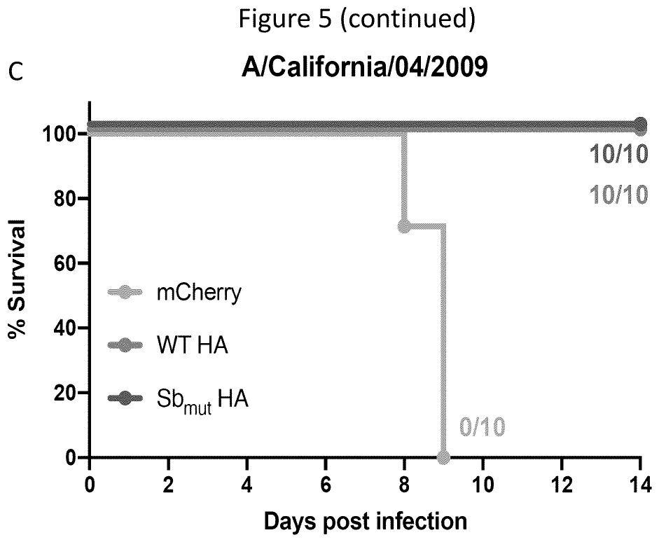

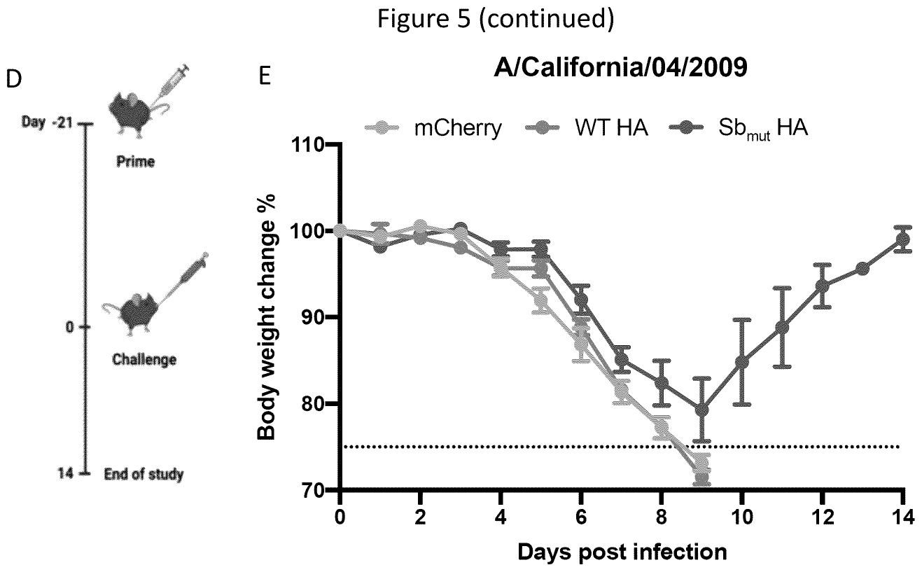

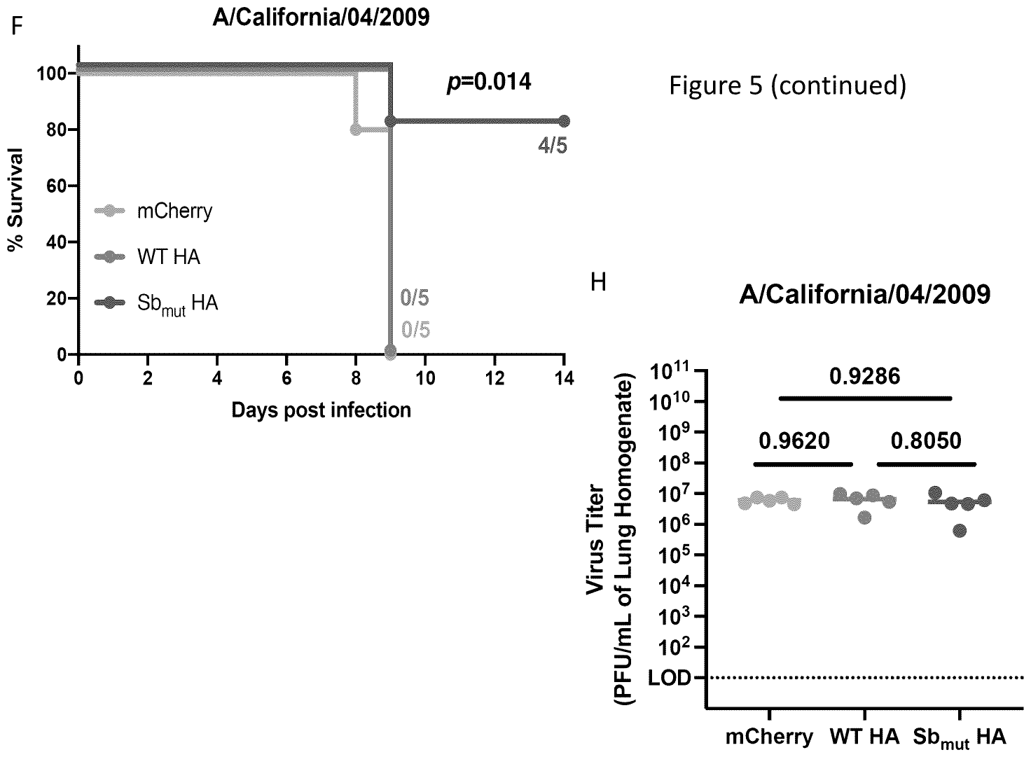

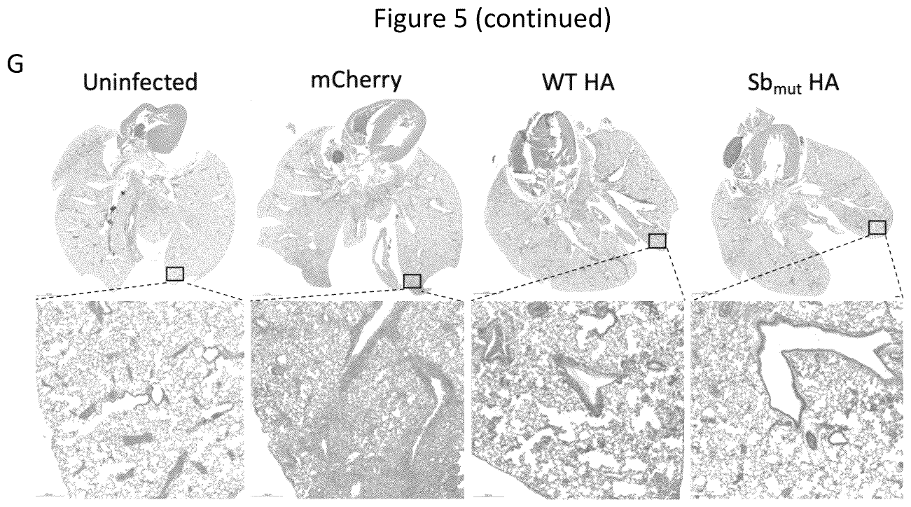

Finally, we wanted to directly test if the improved vaccine responses that we could measure in vitro would translate to improved protection from live virus challenge. After the same adjuvanted prime-boost strategy described above, we challenged with mice with a lethal dose of A/California/04/2009 (FIG. 5A). The mCherry vaccine control group rapidly lost weight after infection, and while both vaccine groups displayed improved protection relative to control, the Sb mutant vaccine group lost significantly less weight than the parental HA group (FIG. 5B). All control vaccinated animals eventually succumbed to infection, while both vaccine groups provided complete protection from mortality (FIG. 5C). Because we could not detect a difference in mortality between the two HA vaccine groups, we repeated the challenge after a single priming vaccination (FIG. 5D). The lack of a boost led to significant weight loss in both of the HA vaccinated groups, however animals vaccinated with the Sb mutant vaccine were able to recover from infection prior to reaching the humane endpoint (FIG. 5E,F). Analysis of lung tissue sections revealed less severe disease in animals vaccinated with the Sb mutant vaccine, however we were unable to detect a change in viral lung titers (FIG. 5G,H). Thus, this vaccine, in the context of infection with a highly antigenically drifted strain, still provides protection from mortality but likely not by directly neutralizing infectious virus.

### REFERENCES FOR THE EXAMPLES

1. Tan G S, Krammer F, Eggink D, Kongchanagul A, Moran T M, Palese P. 2012. A pan-H1 anti-hemagglutinin monoclonal antibody with potent broad-spectrum efficacy in vivo. J Virol 86:6179-88.
2. Luo Z, Girton A W, Heaton B E, Heaton N S. 2021. Engineered influenza virions reveal the contributions of non-hemagglutinin structural proteins to vaccine mediated protection. J Virol doi:10.1128/JVI.02021-20.
3. Whittle J R, Zhang R, Khurana S, King L R, Manischewitz J, Golding H, Dormitzer P R, Haynes B F, Walter E B, Moody M A, Kepler T B, Liao H X, Harrison S C. 2011. Broadly neutralizing human antibody that recognizes the receptor-binding pocket of influenza virus hemagglutinin. Proc Natl Acad Sci USA 108:14216-21.

1. A protein library comprising variants of an antigenic viral protein, wherein each variant of the antigenic viral protein comprises a different set of mutations at one or more hypervariable sites in the antigenic viral protein.

2. The protein library of claim 1, wherein the protein library comprises at least 1×10⁴ variants of the antigenic viral protein.

3. (canceled)

4. The protein library of any one of claim 1, wherein the antigenic viral protein is from influenza virus, human immunodeficiency virus (HIV), severe acute respiratory syndrome coronavirus 2 (SARS-CoV-2), hepatitis C virus (HCV), respiratory syncytial virus (RSV), or human cytomegalovirus (CMV).

5. The protein library of claim 4, wherein the antigenic viral protein is hemagglutinin (HA).

6. The protein library of claim 5, wherein the antigenic viral protein is a group 1 influenza A HA, a group 2 influenza A HA, or an influenza B HA.

7. The protein library of claim 6, wherein the one or more hypervariable sites are in:
a) a region of HA 1 selected from Cb, Sa, Ca2, Ca1, and Sb, as shown in FIG. 11; or
b) a region of HA 3 selected from A, B, C, D, and E, as shown in FIG. 11.

8. The protein library of claim 7, wherein the one or more hypervariable sites are:
a) selected from positions Sb 192, Sb 193, Sb 196, and Sb 198; or
b) in a region of B HA selected from the 120 loop, 150 loop, 160 loop, and 190 helix, as shown in FIG. 11.

9. (canceled)

10. (canceled)

11. A nucleic acid library encoding the protein library of claim 1, wherein each nucleic acid in the nucleic acid library comprising a sequence encoding one of the variants of the antigenic viral protein of the protein library.

12. The nucleic acid library of claim 11, wherein each nucleic acid in the nucleic acid library further comprises a promoter operably linked to the sequence encoding one of the variants of the antigenic viral protein.

13. A vector library comprising the nucleic acid library of claim 11, wherein each vector in the vector library comprises one of the nucleic acids of the nucleic acid library.

14. (canceled)

15. A virus library comprising the nucleic acid library of claim 11, wherein each virus in the virus library comprises one of the nucleic acids of the nucleic acid library.

16. The virus library of claim 15, wherein the virus is selected from the group consisting of influenza virus, HIV, SARS-CoV-2, HCV, RSV, and CMV.

17. (canceled)

18. A vaccine comprising the protein library of claim 1 and an adjuvant.

19. A method of inducing an immune response against the antigenic viral protein in a subject, the method comprising: administering to the subject the protein library of claim 1.

20. The method of claim 19, wherein the immune response against the variants of the antigenic viral protein is altered as compared to the immune response against a wild-type antigenic viral protein.

21. The method of claim 20, wherein a greater number of antibodies recognizing distinct epitopes of the antigenic viral protein are generated in response to the variants of the antigenic viral protein as compared to in response to vaccination with a wild-type antigenic viral protein.

22. The method of claim 20, wherein a greater proportion of antibodies are generated against conserved sites as compared to hypervariable sites within the antigenic viral protein in response to the variants of the antigenic viral protein as compared to in response to vaccination with a wild-type antigenic viral protein.

23. The method of claim 22, wherein the immune response is protective against a highly drifted strain of the virus.

24. (canceled)

25. (canceled)

26. A method of generating the vector library of claim 13, the method comprising:
a) performing saturation mutagenesis on nucleic acids encoding the antigenic viral protein at one or more hypervariable sites of the antigenic viral protein to obtain mutated nucleic acids;
b) cloning the mutated nucleic acids into viral vectors;
c) packaging the viral vectors into viruses;
d) transducing the viral vectors into cells by infecting the cells with the viruses;
e) identifying infected cells that express the antigenic viral protein;
f) cloning the mutated nucleic acids encoding variants of the antigenic viral protein found in the infected cells into an expression vector to generate the vector library.

27. A method of performing saturation mutagenesis on a plurality of target sites within a DNA sequence of interest, the method comprising:
a) designing a plurality of overlapping primers that are complementary to sequences flanking the target sites and comprise mutations at the target sites;
b) performing a recombination reaction in which the primers are annealed to each other to form a set of recombinant primers that each comprise a different set of mutations at the target sites;
c) extending the recombinant primers using a DNA polymerase to form full-length sequences that span the target sites;
d) amplifying the full-length sequences to form an amplification reaction product;
e) purifying the amplification reaction product; and
f) cloning the amplification reaction product into an appropriate vector.

28. The method of claim 27, wherein the mutations comprise NNK for forward primers and/or MNN for reverse primers.

29. (canceled)
